(19) United States
(12) Patent Application Publication
(10) Pub. No.: US 2025/0123269 A1
(43) Pub. Date: Apr. 17, 2025

(54) C3D4 Human Cell Model for MERS and SARS-C0V-2 Infection

(71) Applicant: Hackensack Meridian Health, Inc., Edison, NJ (US)

(72) Inventors: Ching-Wen Chang, Leonia, NJ (US); David S. Perlin, New York, NY (US); Steven Park, Flushing, NY (US)

(73) Assignee: Hackensack Meridian Health, Inc., Edison, NJ (US)

(21) Appl. No.: 18/919,187

(22) Filed: Oct. 17, 2024

Related U.S. Application Data
(60) Provisional application No. 63/544,558, filed on Oct. 17, 2023.

(51) Int. Cl. G01N 33/50; G01N 21/64

(52) U.S. Cl. CPC G01N 33/5023; G01N 21/6428; G01N 2021/6439; G01N 2333/165

(57) ABSTRACT

A robust human cell culture model permissive to both SARS-COV-2 variants and MERS-COV is critical for assessment and validation of antivirals. Human alveolar A549 cells are regarded as a valuable model for respiratory virus infection. SARS-COV-2 uses the angiotensin converting enzyme 2 (ACE2) receptor for viral entry and the transmembrane serine protease 2 (TMPRSS2) to prime the SARS-COV-2 spike protein. By contrast, MERS-COV utilizes the dipeptidyl peptidase 4 receptor (DPP4) to enter the target cells. Three of which are negligibly expressed in A549. Disclosed herein is a generation of a robust human cell model that carries DPP4, ACE2, and TMPRSS2 receptor expressions. By transducing Dpp4 into A549-ACE2plusC3 cells (ACE2+/TMPRSS2+), the resulting cells expressing DPP4, ACE2 and TMPRSS2 (“ACE2plusC3D4”) are highly susceptible to MERS-COV and SARS-CoV-2 omicron infection. This ACE2plusC3D4 cell model can be applied for evaluation of antiviral drugs and potentially developed for high-throughput screening.

### SEQUENCE LISTING

The instant application contains a Sequence Listing which has been submitted herewith and is hereby incorporated by reference in its entirety. Said.xml copy, created on Jan. 15, 2025, is named HMH52, and is 22,169 bytes in size.

### CROSS-REFERENCE TO RELATED APPLICATIONS

The present application claims the benefit of the filing date of U.S. Provisional Application No. 63/544,558 filed Oct. 17, 2023, the disclosure of which is hereby incorporated herein by reference.

### GOVERNMENT STATEMENT

This invention was made with government support under Metropolitan AntiViral Drug Accelerator (MAVDA) awarded by the National Institute of Health (NIH) and the National Institute of Allergy and Infectious Disease (NIAID). The government has certain rights in the invention.

### TECHNICAL FIELD

The present invention is related to a genetically modified cell line and method of using the genetically modified cell line in the study of viruses and antiviral drugs. The genetically modified cell line may be modified to include vectors associated with angiotensin converting enzyme 2 (ACE2), transmembrane serine protease 2 (TMPRSS2), and dipeptidyl peptidase 4 receptor (DPP4). The genetically modified cell line model may be known as ACE2plusC3D4. The genetically modified cell line model is designed to sustain multiple rounds of viral infections, particularly from SARS-COV-2, MERS-COV, SARS coronavirus variants, other human and animal coronaviruses, and the common cold.

### BACKGROUND

The COVID-19 pandemic, caused by the severe acute respiratory syndrome coronavirus 2 (SARS-COV-2) virus, continues to be a global problem, with new variants emerging and breakthrough infections occurring in fully vaccinated individuals. SARS-COV-2 can infect numerous tissue types in the human body and is particularly destructive to lung epithelial cells. Infection can also result in the deregulation of the immune system, leading to acute respiratory distress syndrome and multi-organ failure in severe COVID-19 patients. In addition, SARS-COV-2 is highly contagious and highly transmissible (compared with other SARS viruses), and infectious variants are continuously evolving.

Another highly pathogenic human coronavirus, Middle East respiratory syndrome coronavirus (MERS-COV), was first reported in 2012 and still occurs seasonal outbreaks in the Middle East. The continuation of the spread of highly pathogenic human coronaviruses emphasizes the importance of continued discovery of broad-spectrum antivirals targeting lethal human coronaviruses.

A robust human cell culture model permissive to both SARS-COV-2 variants and MERS-COV is critical for assessment and validation of antivirals. Human alveolar A549 cells are regarded as a valuable model for respiratory virus infection. SARS-COV-2 uses the angiotensin converting enzyme 2 (ACE2) receptor for viral entry and the transmembrane serine protease 2 (TMPRSS2) to prime the SARS-COV-2 spike protein. By contrast, MERS-COV utilizes the dipeptidyl peptidase 4 receptor (DPP4) to enter the target cells. Further, ACE2, TMPRSS2, and DPP4 are crucial receptors used by several highly pathogenic human viruses for cell entry and infection. Three of which are negligibly expressed in A549.

Therefore there is a need in the art for a robust human cell model expressing multiple viral receptors and successfully demonstrating its susceptibility to both lethal coronaviruses and seasonal coronaviruses, namely OC43 and 229E. A new cell model is needed to serve as a valuable tool for the development of pan-CoV antiviral drugs.

### BRIEF SUMMARY

The present disclosure provides solutions to the above and additional improvements in the art. Herein disclosed is a generation of a robust human lung cell model that carries DPP4, ACE2, and TMPRSS2 receptor expressions. By transducing and engineering Dpp4 into A549-ACE2plusC3 cells (ACE2+/TMPRSS2+), the resulting cells expressing various levels of DPP4, ACE2 and TMPRSS2 (“ACE2plusC3D4”) are highly susceptible to MERS-COV and SARS-COV-2 omicron infection. Thus, the novel ACE2plusC3D4 cell model provides an invaluable platform for various applications such as the evaluation of antiviral drugs and potentially developed for high-throughput screening.

To date, no engineered cell lines have been reported that simultaneously express the Ace2, Dpp4, and Tmprss2 receptors. Research has not yet explored the characterization of the three-receptor infection model or its potential application as a tool for studying antiviral treatments.

To address this challenge, created was a novel cellular tool using the genetically modified cell line A549-Ace2-Tmprss2 (Ace2plus). Then introduced was the Dpp4 receptor gene into this line, which was specifically engineered to express various levels of key receptors involved in viral entry. After a thorough evaluation process, identified was a small number of clones from among hundreds for further assessment of their potential as dependable infection models for the human coronavirus family. A549-Ace2-Dpp4-Tmprss2 (ADT) cells are easy to manage as Vero E6 cells and support high levels of human coronavirus replication, including SARS-COV-2, MERS-COV, OC43-CoV, and 229E-CoV, and have a morphology suitable for imaging and CPE-based assays. The cells displayed clear CPE upon SARS-COV-2, MERS-CoV or 229E-CoV infection.

A549-ADT cell line represents a substantial leap forward in the field of virology and antiviral research, offering innovative solutions to critical challenges. Its ability to serve as a single cell line for multiple antiviral targets, coupled with its versatility for high-throughput screening and investigatory tools for studying virus-host interactions, makes it a transformative asset in the fight against highly pathogenic human viruses. This innovative cell line represents a significant breakthrough in virology and antiviral research, and offers a multifaceted solution to the complex issues posed by SARS-COV-2, MERS-COV, and other human coronaviruses.

The disclosed ACE2plusC3D4 model cell line is an immortal cell line that expresses three key human receptors involved in viral entry, ACE2 represented by SEQ ID NO. 1, TMPRRS2 represented by SEQ. ID. NO. 2, and DPP4 represented by SEQ. ID NO. 3. The disclosed ACE2plusC3D4 cell line has been engineered to contain the optimal expression level of each receptor to achieve maximum viral infection, while maintaining the cell. Moreover, using clones from a single cell line allows for homogeneity which significantly reduces variability between experiments. The novel ACE2plusC3D4 cell line provides an invaluable platform for various applications such as the evaluation of antiviral drugs and potentially developed for high-throughput screening.

In one aspect, A549-Ace2-Dpp4-Tmprss2 (ADT) cells were susceptible to pan-human coronaviruses (SARS-COV-2, MERS-COV, OC43-CoV, and 229E-CoV). A549-ADT clone 21 was found as a suitable cellular tool for studying the potency and efficacy of antiviral strategies that target the human coronavirus family. A549-ADT clone 21 was found to support a broad-spectrum coronavirus antiviral drugs. A549-ADT-based viral replication inhibition assays with coronavirus yielded similar half-maximal inhibitory concentration (IC50) values for Nirmatrelvir as VeroE6-based assays and do not require of a P-glycoprotein (P-gp) inhibitor to prevent drug efflux.

In another aspect, a robust human cell model expressing multiple viral receptors and successfully demonstrated its susceptibility to both lethal coronaviruses and seasonal coronaviruses, OC43 and 229E. Also compared were three 3C-like protease inhibitors and showed that Nirmatrelvir possesses better potential than Pomotrelvir, a selective, orally active SARS-COV-2 3CL protease inhibitor, and Ensitrelvir, an oral antiviral drug for COVID-19 currently approved under the emergency regulatory approval system, for the development of a pan-coronavirus antiviral drug. Furthermore, evaluated were antimalarial drugs and identified were the antiviral activity of Halofantrine against SARS-COV-2. The findings indicate that this new cell model can serve as a valuable tool for the development of pan-CoV antiviral drugs.

The novelty of the genetically modified cell line model, ACE2plusC3D4 or A549-Ace2-Dpp4-Tmprss2 (ADT), having an expression of 3 viral receptors to enhance viral entry also includes the fine tuning of the expression levels of each receptor for optimal viral entry and infectivity. Such fine tuning for a cell line used for antiviral drug screening against pan-coronaviruses is a novel feature created by the present investigators, and has neither been taught nor suggested by others. Current scientific literature does not clearly demonstrate that higher expression of viral entry receptors like DPP4 and ACE2 directly corresponds to increased MERS viral infectivity and pathogenicity, namely, more receptor expression does not equate to better experimental outcomes. Especially for the purposes of drug screening and studying the underlying pathogenicity of viruses for which these cells will be used, needed is an optimal and specialized viral infection levels, where viral entry causes a strong infection without causing rapid cell-death allowing for investigations on biology and recovery following infection. For instance, among the 28 clones of cells expressing the 3 receptors that were examined herein, clones (clones #21, 22, 23, and 24) exhibiting relatively low Dpp4 expression levels (1.7 and 1.9 times greater than the control) and remained susceptible to MERS-COV and SARS-COV2 infection. These particular clones at the above levels were more appropriate for immunofluorescence tests used for antiviral drug screening, in comparison to clones with over 2-10 fold increase in DPP4 expression (clones #1-20). The present investigators also noted that higher Dpp4 expression leads to syncytial cell death due to the increased expression of the receptor itself rather than due to infection.

Furthermore, the present investigators demonstrated that higher Ace2 expression level in clone 21 enabled susceptibility to recent SARS-COV2 omicron subvariants like EG5.1, JN.1, and coronavirus that cause common cold such as OC43-CoV and 229E-CoV (FIG. 19G and FIG. 19H). Additionally, evaluated was its susceptibility to NL63-CoV, but the infection rate was not as high as that observed with OC43-CoV and 229E-CoV. These studies highlight the novelty of optimizing the expression of viral entry receptors for the development of a genetically modified stable cell line ideal for studying pan-coronaviruses and screening antiviral drug candidates.

These and other aspects are further explained in the attached drawings and following detailed description.

### BRIEF DESCRIPTION OF THE DRAWINGS

The patent or application file contains at least one drawing executed in color. Copies of the patent or patent application with color drawing(s) will be provided by the Office upon request and payment of the necessary fee.

FIG. 1A-E illustrates the process and quantification of SARS-COV-2 infection of ACE2plus cells.

FIG. 2A-C illustrates the characterization of single-cell clones from the ACE2plus cell population.

FIG. 3A-G illustrates the characterization of the ACE2plus and ACE2plusC3 cell lines.

FIG. 4 depicts data relating to ACE2plusC3 cells infected with SARS-COV-2 Spike-pseudotyped lentivirus.

FIG. 5A-F depicts data relating to the antiviral activity in ACE2plusC3 cells.

FIG. 6 further depicts data relating to the antiviral activity in ACE2plusC3 cells.

FIG. 7 illustrates an abbreviated family tree of known human coronaviruses.

FIG. 8 is a schematic of the immunofluorescence-based assay to examine anti-MERS-COV activity in engineered A549 cells.

FIG. 9 illustrates the measuring ability of compound to reverse the viral induces cytopathic effect in engineered A549 cells.

FIG. 10 illustrates data relating to the permissibility of the engineered A549 cells.

FIG. 11 illustrates results of immunofluorescence staining of the nucleocapsid protein of MERS-COV and SARS-COV-2.

FIG. 12 illustrates a MERS-COV Spike-induced cell fusion after infection.

FIG. 13 illustrates results relating to the permissibility of the engineered A549 cell line for MERS-COV infection.

FIG. 14 is a schematic of the immunofluorescence-based and cytopathic effect (CPE)-based assay to examine anti-MERS-COV activity in engineered A549 cells.

FIG. 15 further illustrates the measuring ability of compound to reverse the viral induces cytopathic effect in engineered A549 cells.

FIG. 16 illustrates the workflow of high throughput screening of antiviral drugs.

FIG. 17 is a schematic overview of the higher throughput assay.

FIGS. 18A-18C show one embodiment of an establishment of a new cellular tool for human coronavirus infection. FIG. 18A is a schematic illustration of the procedure to generate MERS-COV and SARS-COV-2 susceptible cell lines. FIG. 18B is MERS entry receptor, Dpp4 expression level is correlated to virus-induced cell fusion. Images showing anti-NP staining (viral infection, red or light shading) Hoechst staining (nuclei, blue or dark shading), 48 h post infection. Scale bar 200 μm. FIG. 18C illustrates Dpp4 and Ace2 receptors are co-expressed in A549-Ace2-Dpp4-Tmprss2 cells.

FIGS. 19A-19H show characterization of A549-Ace2-Dpp4-Tmprss2 subclones. FIG. 19A illustrates Dpp4 and Ace2 receptor intensity of twenty-eight clones were analyzed by flow cytometry. FIG. 19B illustrates the level of MERS-induced CPE from each clone, which expressed different level of Ace2 and Dpp4, was analyzed under microscope and plotted with Dpp4 (left) (FIG. 19B(1)), Ace2 (right) (FIG. 19B(2)) intensity, respectively. FIG. 19C illustrates a heatmap representing normalized CPE level and receptor intensity of twenty-eight clones, on a scale 0 to 1. Relative expression of Tmprss2 gene in each clone was analyzed by qPCR. FIG. 19D illustrates a bright-field images of mock-vs virus-infected cells. FIG. 19E illustrates Cell viability (left), viral-induced CPE in selected clones were determined by CellTiter Glo assay, and FIG. 19E illustrates virus infectivity (right) determined by viral nucleocapsid protein expression and normalized to DMSO control without virus infection. FIG. 19G are images of cells (mock, non-infected control), omicron subvariant, cold coronavirus infected cells (red or light shaded, anti-NP staining; blue or dark shaded, nuclei). Scale bar 200 μm. FIG. 19H illustrates the percentage of each virus infection in clone 21 calculated as described in materials and methods, herein.

FIGS. 20A-20B show an assessment of the antiviral activity of nirmatrelvir in selected clones. FIG. 20A illustrate Cells were infected with MERS at an MOI of 0.1 for 48 h in the presence of the indicated concentrations. For IF assay, infection was calculated by dividing the number of infected cells (measured by MERS nucleocapsid protein) by the total cell nuclei present and normalized to DMSO control group. The red line or light shaded line indicates the inhibition of MERS infection. In parallel, CPE reduction assay was carried and showed as blue line or darker line. The curves were fitted using GraphPad Prism software. Half maximal inhibitory concentration (IC50) was calculated from the curve fit. FIG. 20B illustrate the antiviral activity of nirmatrelvir in VeroE6-Tmprss2 cells were measured with or without P-gp inhibitor.

FIGS. 21A-21B show the following. FIG. 21A illustrates graphs showing Application of A549-Ace2-Dpp4-Tmprss2 cells to evaluate antivirals against hCoVs. A549 cells were seeded in 96-well plate and infected with indicated coronaviruses at a multiplicity of infection of 0.1 and treated with serially diluted compounds, PF-07321332, PBI-0451, and S-217622. Infected cells were fixed at 48 h post infection and used for immunofluorescence stain. Data are shown percent of virus infectivity in compound-treated cells relative to DMSO vehicle control-treated cells. Data are representative of the average independent experiments. FIG. 21B is a Table showing IC50 values of compounds on inhibition of various human coronavirus replication activity.

FIGS. 22A-22D show screening the activity of the repurposed antimalarials against SARS-COV-2 in A549-Ace2-Dpp4-Tmprss2 cells. FIG. 22A is a schematic of screening strategy used for the repurposed drugs analysis. FIG. 22B shows antimalarial drugs that were evaluated for the ability to inhibit MERS-induced CPE and cell death. FIG. 22C illustrates antimalarial compounds that were evaluated for the ability to inhibit virus replication, 48 h post infection. FIG. 22D illustrates drug cytotoxicity that was measured with CellTiter Glo assay, 3 days post treatment.

FIGS. 23A-23B show anti-omicron-JN.1 activity of selected drugs in A549-Ace2-Dpp4-Tmprss2 cells. FIG. 23A are graphs illustrating concentration-dependent inhibition of the replication of JN.1 in infected cells by halofantrine, amodiaquine, and chloroquine as determined by HCl at 48 h. Red or light shaded curves represent the percentage of virus infection related to uninfected DMSO-treated cells. Blue curve or dark shaded curves represent cell viability after 72 h at the same concentrations of test compounds. Representative images and chemical structure of the drugs are also presented. FIG. 23B is a table illustrating anti-coronavirus profile of test MMV compounds in A549-ADT cells.

FIGS. 24A-24B show an analysis of the cell surface expression of Ace2 and Dpp4 receptors in A549-Ace2-Dpp4-Tmprss2 subclones. FIG. 24A illustrates an overlay of the MFI histograms obtained by flow cytometry analysis. The subclones showed varied Dpp4 receptor intensity. FIG. 24B illustrates bar graphs of the mean fluorescent intensity (MFI) values obtained after quantification of the fluorescent signal.

### DETAILED DESCRIPTION

### 1. Introduction

Human Dipeptidyl Peptidase-4 (DPP4) is a single pass transmembrane protein with multiple functions on glycemic control, cell migration and proliferation, and the immune system, among other systems. It has been demonstrated that acts as a receptor for MERS-COV. Angiotensin-converting enzyme 2 (ACE2) is an enzyme that can be found either attached to the membrane of cells (mACE2) in the lung, intestines, kidney, testis, and gallbladder. Human ACE2 may also serve as the entry point into cells for some coronaviruses, including HCoV-NL63, SARS-COV, and SARS-COV-2.

Transmembrane serine protease 2 (TMPRSS2) is an enzyme that in humans is encoded by the TMPRRS2 gene. It belongs to the TMPRSS2 family of proteins, whose members are transmembrane proteins which exhibit serine protease activity. The TMPRSS2 protein is found in high concentration in the cell membranes of epithelial cells of the lung of the prostate, but also in the heart, liver, and gastrointestinal tract. Some coronaviruses, such as SARS-COV-1, MERS-COV, and SARS COV-2, are activated by TMPRRS2 and can be inhibited by TMPRRS2 inhibitors.

The infectivity of SARS-COV-2 strongly depends on ACE2, a host factor. SARS-COV-2 uses the SARS-COV receptor ACE2 for entry. Similarly, MERS-COV uses the Dpp4 receptor for viral entry. The serine protease TMPRRS2 acts as co-receptors in the process of virus entry.

The SARS-COV-2 spike(S) glycoprotein interacts with the host ACE2 receptor to initiate S1-S2 cleavage by TMPRSS2. Furthermore, endosomal proteases release the viral RNA genome into the host cell. Thus, the internalized viral RNA utilizes the host's cellular protein machinery to counter antiviral responses in the host cell. Developing a robust in vitro system for studying SARS-COV-2 requires a cell line that accurately models these events.

Although human stem cell-based lung epithelial models are available for studying SARS-COV-2, they require advanced expertise in cell culture and are costly to propagate for large-scale screening applications. Alternative cell lines, such as human lung-derived Calu-3 and colon-derived Caco-2 cells, are permissive to SARS-COV-2 infection and show virus replication over a period of 120 h. However, neither cell line shows comparable infectivity to Vero E6 cells or displays SARS-COV-2-induced substantial cell damage and cytopathic syncytia formation, emphasizing the need for an improved platform for studying SARS-COV-2 biology and identifying antivirals.

Lentiviral transduction is an efficient method of expressing transgenes in mammalian cells through stable chromosomal integration; however, gene integration is random and generates a heterogeneous population of transduced cells. In previous studies, lentiviral methods were used to generate A549 cells (43.20) expressing ACE2 and TMPRSS2. The susceptibility of SARS-COV-2 infection in 43.20 cells was dramatically reduced after several rounds of sequential culture. This observation might be associated with the reduced ACE2 expression level in 43.20 cells. Intriguingly, Sherman et al. recently reported that the expression of ACE2 on the cell surface is heterogeneous in Huh-7 and Calu-3 cell lines. They found that ACE2 expression was unstable after sorting, and the proportion of positive cells gradually reverted to the parental distribution after several passages. In other words, the expansion of ACE2-positive cells and cellular ACE2 surface expression increased after sorting but subsequently decreased over multiple generations of daughter cells. This phenomenon is similar to what we observed in the 43.20 cell model.

Herein the development of a robust A549-based cell line is shown that highly expresses ACE2 and TMPRSS2 and remains relatively stable to SARS-COV-2 infection susceptibility after multiple passages, providing a valuable cell line for performing high-throughput in vitro testing to evaluate the efficacy of SARS-COV-2 antivirals and facilitate research on drugs for COVID-19 treatment.

Newly engineered A549 models are highly permissive to MERS-COV, SARS-CoV-2 infection, including emerging omicron variants EG5.1, BQ1, XBB1.19 and XBB1.16. This engineered A549 cell model shows significant cytopathic effects (CPE) and virus-induced CPE can be rescued by adding Niamatrelvir during virus infection. This novel A549 cell model shows its potential for antiviral drug discovery, further condition optimization for integrating this model into our current HTS assay is ongoing. MERS-COV shows faster virus replication kinetics than SARS-COV-2 in the engineered A549 cell model, suggesting that this model can be used for studying coronavirus pathogenesis.

Current available cell lines used for both SARS and MERS-COV are primary cells. Primary cells are mortal cells obtained from live tissue samples. There are significant disadvantages associated with using primary cells for antiviral drug screening assays. There is limited availability of primary cells. Primary cells are usually obtained directly from tissues or organs, and their supply is finite. This limitation can be a significant challenge for conducting high-throughput drug screening assays. There is variability between donors of primary cells. Primary cells sourced from different donors can exhibit substantial inter-individual variability in terms of their response to viral infections and antiviral drugs. This variability can complicate experimental results and make it challenging to draw general conclusions. Primary cells have a short lifespan. Primary cells have a limited lifespan in vitro, and they tend to lose their functionality and characteristics over time. This can hinder the feasibility of conducting long-term antiviral drug screening studies. There are senescence and differentiation issues associated with primary cells. Primary cells may undergo senescence or differentiate into other cell types during culture, making it difficult to maintain a stable cell population with consistent characteristics over time.

There are ethical and logistical challenges in using primary cells. Obtaining primary cells may require invasive procedures, and ethical considerations may limit their use, particularly when sourcing them from human donors. Additionally, the coordination of collecting and maintaining primary cells can be complex. There is a lack of immortalization with primary cells. Primary cells cannot be infinitely propagated in culture because they lack the ability to undergo unlimited replication. This limitation can restrict their utility for long-term and large-scale antiviral drug screening efforts. Primary cells are inherently heterogeneous, containing various subpopulations with different properties. This heterogeneity can make it challenging to control for variables and obtain consistent results. Primary cells are often challenging to genetically manipulate, making it difficult to introduce specific mutations or changes to study their effects on antiviral drug responses. Some primary cell types, especially those relevant to specific tissues or diseases, may be challenging to obtain or culture, limiting their use in certain antiviral drug screening studies. Primary cell culture can be expensive and resource-intensive, as it often requires specialized equipment, culture media, and facilities, adding to the overall cost of drug screening experiments.

One of the major challenges in creating an engineered cell line for drug screening efforts is optimizing expression of the DPP4 receptor for maximum viral infection and replication. Although the human DPP4 sequence is publicly available, the unique insertion site of the DPP4 receptor gene will affect the expression level of the protein.

The ACE2plusC3D4 cell line represents a significant breakthrough in virology and antiviral research. ACE2, TMPRSS2, and DPP4 are crucial receptors used by several highly pathogenic human viruses for cell entry and infection. This novel cell line provides an invaluable platform for various applications.

ACE2plusC3D4 may be used as a single cell line for multiple antiviral targets. The ACE2plusC3D4 cell line serves as a versatile platform for antiviral drug development. It can be used to screen and evaluate potential antiviral compounds against a wide range of viruses that utilize ACE2, TMPRSS2, and DPP4 receptors for entry including SARS, MERS, and common cold viruses. Researchers and pharmaceutical companies can use this cell line to assess the efficacy of antiviral agents against different virus strains, including novel variants. This reduces the need for multiple cell lines and simplifies the drug development process. Use of a single cell line reduces cost and resources associated with individual protocols for multiple cell lines.

ACE2plusC3D4 may be used as an investigatory tool for studying virus-host interactions. The ACE2plusC3D4 cell line is a valuable tool for researchers studying virus-host interactions. It allows for in-depth investigation into the mechanisms of viral entry, host cell responses, and the development of new therapeutic strategies. This cell line provides insights into how various coronaviruses, as well as other viruses that may use similar receptors, interact with host cells. Such knowledge is critical for understanding pathogenesis and developing targeted interventions.

ACE2plusC3D4 may be used as an in vitro co-infection model. The ACE2plusC3DPP4 cell line is ideally suited for studying co-infections. It can be co-infected with multiple viruses, simulating complex scenarios seen in clinical cases. This in vitro co-infection model enables the evaluation of interactions between different viruses and their impact on host cells, as well as the potential development of antiviral therapies capable of addressing co-infections effectively.

ACEplusC3D4 may be used to identify broad-spectrum antiviral agents. Through the constructive collaboration testing of antiviral drug combinations using the ACE2plusC3D4 cell line, it becomes possible to identify and develop broad-spectrum antiviral agents capable of targeting multiple viruses (broad-spectrum antiviral activity) simultaneously.

Low-passage SARS-COV-2 stocks were used in this study. USA-WA1/2020 strain (BEI #NR-52281) was propagated by infecting Vero E6 cells at a low multiplicity of infection (MOI) and then maintaining the infected cells in Eagle's Minimal Essential Medium (EMEM, Millipore Sigma, Burlington, MA, USA) with 2% fetal bovine serum (FBS, Atlas Biologicals, Fort Collins, CO, USA). Cell-free virus supernatant was harvested 72 h post-infection when <70% cytopathic effects were evident. Aliquots of concentrated virus stocks were stored at −80° C., titered by tissue culture infectious dose (TCID50), and used to infect the different cell lines at an MOI of 0.1 or 0.4. An mNeonGreen-labeled infectious clone-derived strain (icSARS-COV-2-mNG) was received from the World Reference Center for Emerging Viruses and Arboviruses at the University of Texas Medical Branch after obtaining a material transfer agreement between the two institutions. The virus was reconstituted and propagated according to the previously described method. Briefly, the lyophilized virus was reconstituted with 0.5 mL of sterile water, and aliquots of 25 μL and 50 μL were stored in a −80° C. freezer. An aliquot of the reconstituted virus was used for a low MOI infection of ˜90% confluent Vero E6 cells. The infected cells were maintained in EMEM with 2% FBS, and the cell-free virus supernatant was harvested, stored, and titered as described for the WT virus. The two variants, B.1.617.2 delta (BEI #NR-55672) and B.1.1.529 omicron (BEI #NR-56461), were propagated by infecting Vero E6 TMPRSS2-T2A-ACE2 cells at a low MOI 0.01 and maintained in EMEM with 2% FBS. The cell-free virus supernatant was harvested, stored, and titered as described for the WT virus. All work with infectious SARS-COV-2 was performed in a biosafety level 3 laboratory facility (BSL-3) by personnel trained to manage BSL-3 agents according to standard operating procedures and approved by the University of Massachusetts Chan Medical School Institutional Biosafety Committee.

A549 (CCL-185), Calu-3 (HTB-55), and Vero E6 (CRL-1586) cells were obtained from ATCC. Vero E6-TMPRSS2-T2A-ACE2 (NR-54970) cells were provided by BEI Resources. Commercial cell lines A549-hACE2 (a549-hace2, lot 42-01) and A549-hACE2-TMPRSS2 (a549-hace2tpsa, lot 42-02) were purchased from InvivoGen in February 2021. A549-43.20 cells were generated by Dr. Machr's laboratory as described in Koupenova et al. Cells were cultured at 37° C. in Dulbecco's Modified Eagle Medium (DMEM) supplemented with 10% heat-inactivated FBS, 100 U/mL penicillin-streptomycin, and 1% L-glutamine. The ACE2plusC3 cell line is provided by the present investigators, and may also become available from the ATCC (CRL-3560) when the present investigators send the cell line to ATCC.

The plasmid sets for generating the SARS-COV-2 S pseudotyped virus were provided by BEI Resources (NR-52948; NR-53765). The generation of pseudotyped lentiviral particles was based on the previously described protocol. The pHAGE-EFla-ACE2-PGK-puroR and pHAGE-EFla-TMPRSS2-PGK-puroR plasmids were used to establish A549-43.20. Anti-ACE2 and anti-TMPRSS2 antibodies were purchased from R&D Systems (Minneapolis, MN, USA, Catalog No. AF933) and Santa Cruz Biotechnology, Inc. (Dallas, TX, USA, Catalog No. sc-515727), respectively.

Decanoyl-RVKR-CMK (#3501) was purchased from TOCRIS (Bristol, UK). Camostat mesylate (SML0057) and naphthofluorescein (#70420) were purchased from Millipore Sigma (Burlington, MA, USA). Nirmatrelvir (HY-138687), EIDD-1931 (HY-125033), remdesivir (HY-104077), nelfinavir mesylate (HY-15287A), E64d (HY-100229), and fluvoxamine (HY-B0103) were purchased from MedChemExpress (Monmouth Junction, NJ, USA). Dimethyl sulfoxide (DMSO) was purchased from Sigma-Aldrich (St. Louis, MO, USA, Catalog No. D8418).

### 2.2. RNA Isolation and RT-PCR

RNA was isolated from virus-containing medium or cell lysates using TRIzol LSTM and stored at −80° C. for RT-qPCR. The expression level of isolated viral RNA was quantified using the QuantiFast Pathogen RT-PCR Kit (Qiagen, Germantown, MD, USA, Catalog No. 211352) and the 2019-nCOV RUO Kit (IDT, Redwood City, CA, USA, Catalog No. 10006713). The cycling conditions followed the manufacturer's protocol. Isolated cellular RNA was incubated in gDNA Wipeout Buffer to remove contaminating genomic DNA. The purified RNA was then used for cDNA synthesis using the QuantiTect Reverse Transcription Kit (Qiagen, 205311). The resultant cDNA was used to measure mRNA expression levels of ACE2 and TMPRSS2 by qPCR with gene-specific primers (human ACE2, sense 5′-GGGATCAGAGATCGGAAGAAGAAA-3′, [SEQ.4] and antisense 5′-AGGAGGTCTGAACATCATCAGTG-3′ [SEQ.5]; human TMPRSS2, sense 5′-AATCGGTGTGTTCGCCTCTAC-3′ [SEQ. 6], and antisense 5′-CGTAGTTCTCGTTCCAGTCGT-3′ [SEQ. 7] and Applied BiosystemsTM SYBR Green reagent (Thermo Fisher Scientific, Waltham, MA, USA, Catalog No. 4309155). GAPDH was used as an endogenous control gene (sense 5′-TCCTCCACCTTTGACGCT-3′ [SEQ. 8] and antisense 5′-TCTTCCTCTTGTGCTCTTGC-3′ [SEQ. 9]).

### 2.3. Plaque Assay

Approximately 2×105 Vero E6 cells were seeded in each well of 12-well plates and grown at 37° C. in 5% CO2 for 18 h. The virus was serially diluted in 1× Minimum Essential Media (MEM) with 3% FBS. Cell monolayers were aspirated and inoculated with 300 μL of virus inoculum. The infected cells were incubated at 37° C. with 5% CO2 for 1 h. The virus-containing medium was then aspirated from the cells and replaced with an overlay medium containing 1×MEM with 0.42% bovine serum albumin (BSA), 20 mM HEPES, 0.24% NaHCO₃, and 0.7% agarose (Thermo Fisher Scientific, Waltham, MA, Catalog No. LP0028). After a 72-h incubation, the cells were fixed with 4% paraformaldehyde (PFA) overnight and then stained with crystal violet solution (Sigma-Aldrich, St. Louis, MO, USA) and quantified.

### 2.4. Immunofluorescence Staining, Cell Sorting, and Flow Cytometry

Cells were plated on 96-well tissue culture plates (Corning Inc., Corning, NY, USA) and infected with low-passage SARS-COV-2 or icSARS-COV-2-mNG at the indicated MOIs and incubated for designated times. Infected cells were fixed with 4% PFA for 30 min at room temperature, gently washed twice with phosphate-buffered solution (PBS), and then permeabilized with 1% Triton X-100 in PBS. The cells were then blocked with 5% BSA. The fixed cells were treated with either a human monoclonal primary antibody conjugated with Alexa-488 targeting the SARS-COV-2 S antigen at 1:200 dilution or a mouse monoclonal primary antibody targeting the nucleocapsid protein (NP) antigen (SinoBiological, Beijing, China, Catalog No. 40143-MM08) at 1:1000 dilution and incubated for 2 h at 4° C. After washing the cells with 0.05% Tween 20 solution, they were treated with an anti-mouse goat secondary antibody conjugated with Alexa-594 (Invitrogen, Carlsbad, CA, USA, Catalog No. A-11005) at 1:2000 dilution and incubated for 1 h at 4° C. The cells were then counterstained with 4′,6-diamidino-2-phenylindole (DAPI; Abcam, Cambridge, MA, USA) for 15 min at 4° C. to visualize the cell nuclei. Images were acquired with Celigo Image Cytometer or ImageXpress Micro-XL (IXM) system by immunofluorescence with 4× and 10× objectives. The images were processed by Celigo Image Software (Nexcelom Bioscience, Lawrence, MA, USA, version 200-BFFL-5C) or Meta-Xpress Software (Molecular Devices, San Jose, CA, USA, version 4.0.0.43).

To increase the infectivity of the cells, the ACE2+ populations were further sorted using an ACE2-specific antibody to establish an ACE2plus cell line and its subclones. Briefly, trypsinized cells were stained with goat anti-ACE2 antibody (R&D, AF933) and sorted using a BD FACSII sorter (BD Biosciences, Franklin Lake, NJ, USA), located in the Flow Cytometry Core, UMass Chan Medical School. For flow cytometric analysis, freshly trypsinized cells were stained with goat anti-ACE2 antibody (R&D, AF933) on ice for 30 min. After wash, cells were incubated with Alexa-647-conjugated donkey anti-goat IgG antibody (A-21447, Invitrogen) on ice for a half hour, then washed and resuspended in an appropriate volume of cell-sorting buffer (1×PBS, 0.5% BSA, 2 mM EDTA). Flow cytometric analysis was performed using a BD LSRII Flow Cytometer.

### 2.5. Luciferase Assay and Cytotoxicity

SARS-COV-2 S pseudotyped virus activity was determined using the Bright-Glo Luciferase Assay System (Promega, Madison, WI, USA). Luminescence was quantified at 48 h post-virus (or mock) infection of the ACE2plus cells. Cell death was measured using the LDH Cytotoxicity Detection Kit from Roche (11644793001) to measure the lactate dehydrogenase activity in the supernatants.

### 2.6. Statistical Analyses

Data are expressed as means±standard deviations (SD), and the significance of differences between groups was evaluated using ANOVA with Dunnett's multiple comparisons test. All tests were performed using Prism 9 (GraphPad Software, v9).

### 3. Results

3.1. Establishing an ACE2plus Cell Line that is Highly Permissive to SARS-COV-2 Infection

To establish a robust and stable human lung alveolar cell line for SARS-COV-2 studies, we transduced A549 cells with human ACE2-expressing lentivirus at a low MOI and selected them with puromycin (1 μg/mL).

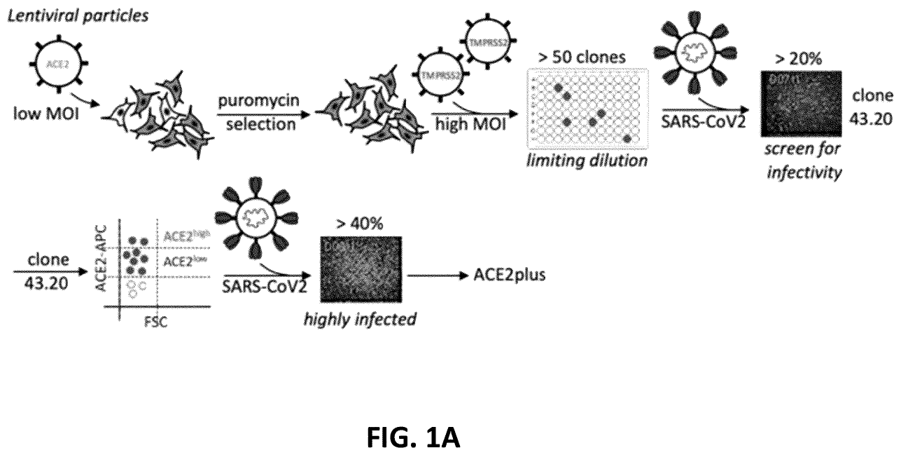

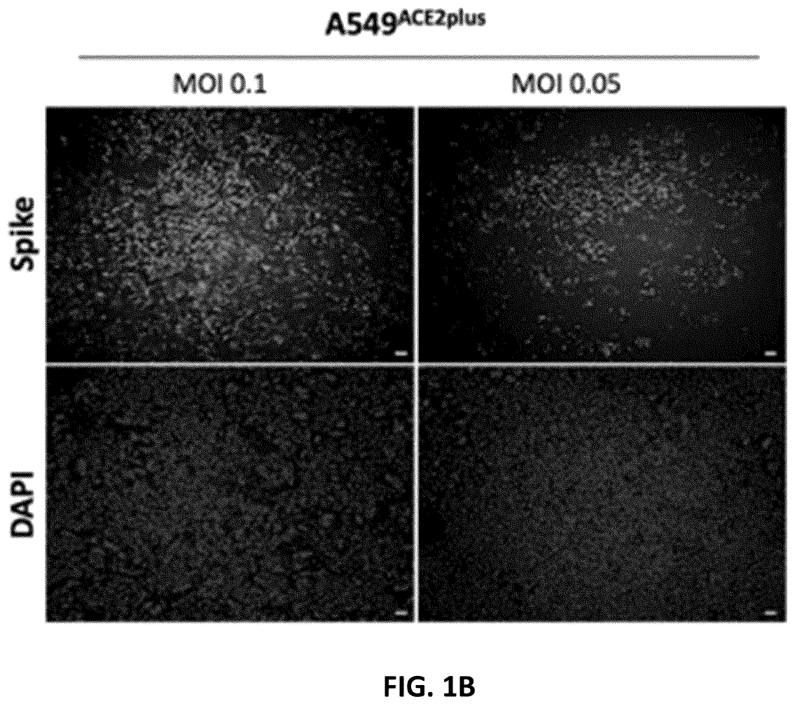

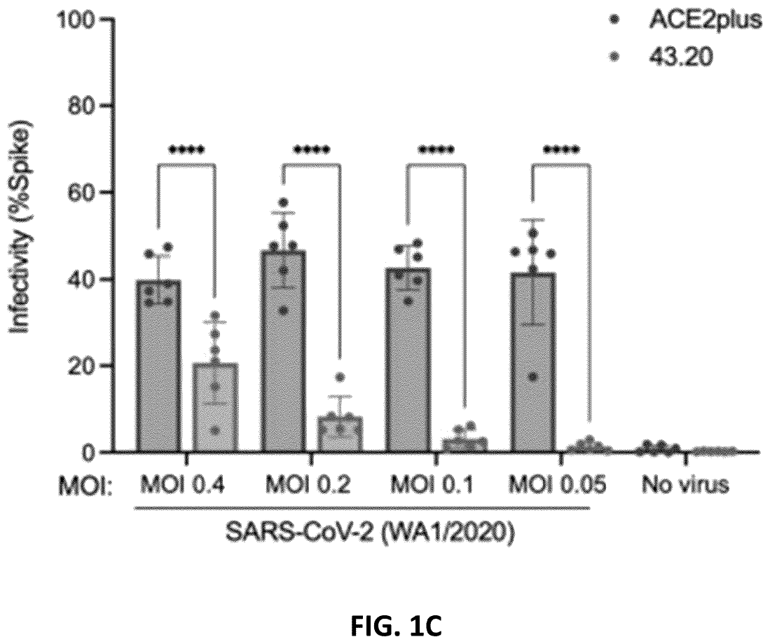

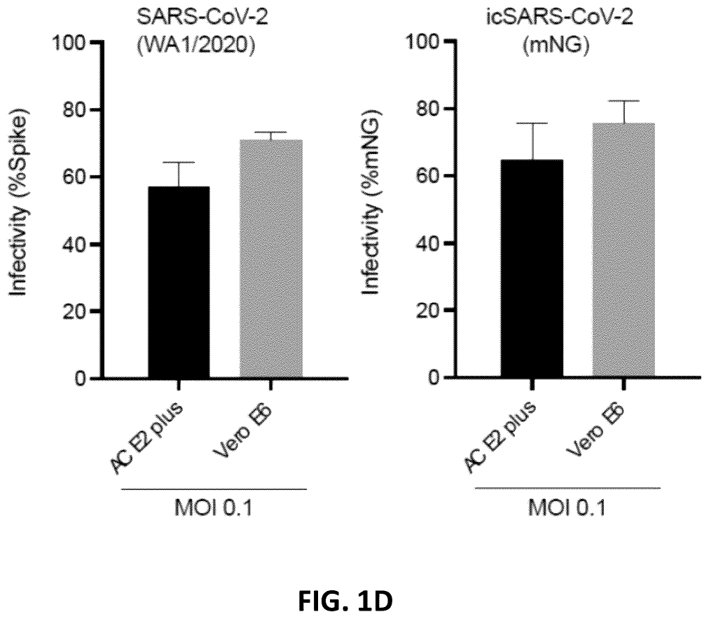

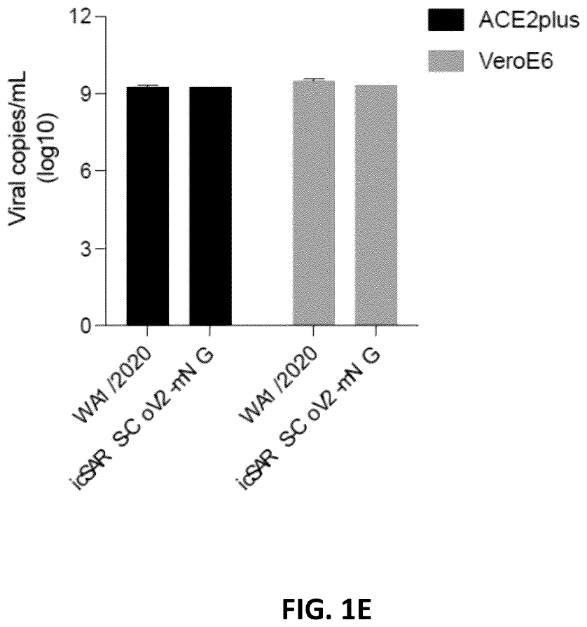

FIGS. 1A-C illustrate the process of creating and confirming a highly permissive ACE2plus cell line for SARS-COV-2 infection. FIG. 1A depicts the experimental scheme to establish A549-ACE2-TMPRSS2 (43.20) and A549-ACE2-TMPRSS2 (ACE2plus) cells. As described in and shown in FIG. 1A, puromycin-resistant cells were further transduced with human TMPRSS2-expressing lentivirus at a high MOI. Following limiting-dilution cloning, over 50 clones were selected and challenged with SARS-COV-2 USA-WA 1/2020 (referred to as “wild type”, WT). Of the 50+clones, clone 43.20 exhibited the most efficient SARS-COV-2 infection rate, which was further enriched by cell sorting with an ACE2-targeting antibody as described in Materials and Methods. FIG. 1B displays, immunofluorescence staining of the spike protein of SARS-COV-2 at 48 h post-infection. Scale bar, 100 μm. FIG. 1C illustrates the increased infectivity of ACE2plus cells. Wherein, the percentage of infected cells was quantified using ImageXpress to determine the infectivity (spike+ cells in total cell number). Data are expressed as mean±SD. n=6 biological replicates. ****, p<0.0001. As shown in FIGS. 1B and 1C, following another round of puromycin selection (5 μg/mL), the infectivity significantly increased to ˜40%. Next, we compared the infectivity of ACE2plus and Vero E6 cells. Each cell type was challenged with WT SARS-COV-2 or the mNeonGreen-labeled SARS-COV-2 strain icSARS-COV-2mNG (WT-mNG) at MOIs of 0.05 and 0.1 for 48 h (control cells received growth medium alone).

FIGS. 1D-E depict the ACEplus cells show comparable infectivity to Vero E6 cells. Cells were challenged with SARS-COV-2 (WA1/2020) or icSARS-COV-2-mNG (mNG) for 48 hours, then the percentage of infected cells was quantified by ImageXpress to measure the infectivity. As shown in FIG. 1D, the ACE2plus cells exhibited infection rates of ˜60%, similar to the infection rate in the Vero E6 cells (˜70%). Virus-containing supernatants from infected ACE2plus and VeroE6 cells were collected and analyzed by RT-qPCR to determine the viral copy number. The data represent the mean±SD. As shown in FIG. 1E, the two lines showed similar viral nucleoprotein transcript levels in the supernatants.

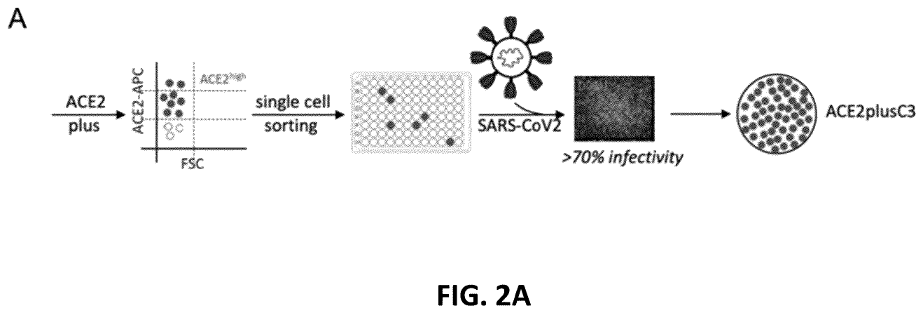

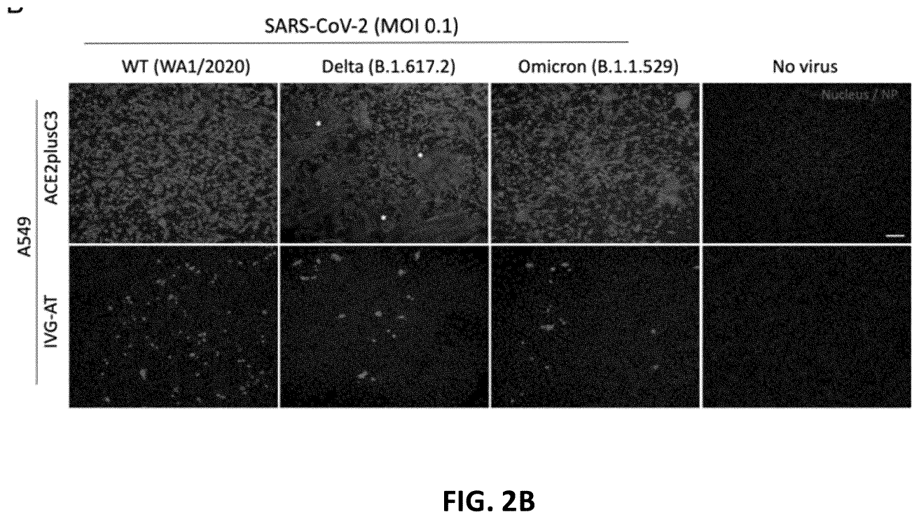

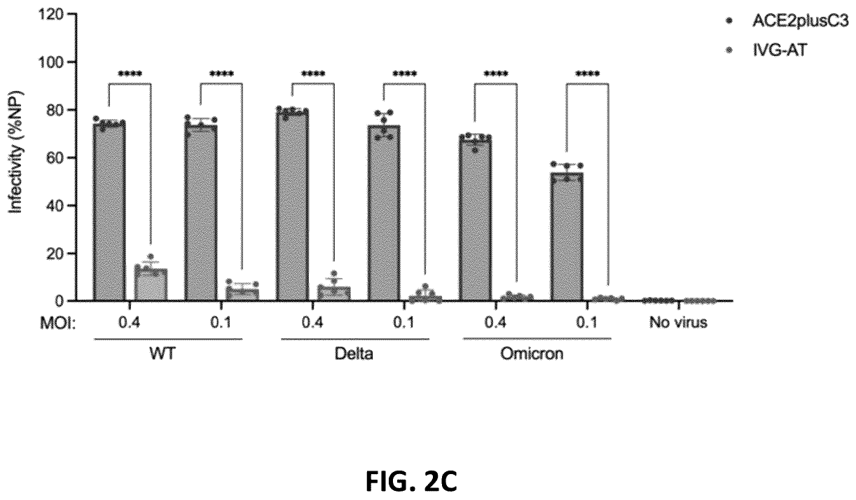

FIGS. 2A-C illustrate the characterization of single-cell clones from the ACE2plus cell population. The experimental scheme to establish the ACE2plusC3 line. The commercial A549-hACE2-TMPRSS2 cell line (IVG-AT) was evaluated using the same experimental conditions. Shown in FIG. 2A, a single-cell sorting of ACE2plus cells generated single-cell-derived ACE2plus subclones. Over 20 clones were isolated and challenged with WT SARS-COV-2, with most clones showing >60% infectivity. To evaluate its susceptibility to different SARS-COV-2 variants, we infected ACE2plusC3 cells with the WT virus or the delta and omicron variants and compared them with commercial A549-hACE2-TMPRSS2 cells (“IVG-AT”) used by researchers studying SARS-COV-2. As shown in FIG. 2B, viral nucleocapsid protein (NP) was detected in most ACE2plusC3 cells 48 h post-infection. Virus-infected cells are visualized by immunofluorescence staining of the nucleocapsid protein of SARS-COV-2 at 48 h post-infection. Scale bar, 200 μm. Syncytia are indicated by asterisks. However, NP signals were sparse in IVG-AT cells, and we rarely observed virus-induced cell fusion. In FIG. 2C, ACE2plusC3 cells showed superior infectivity with WT SARS-COV-2 and the delta and omicron variants. The percent infectivity is determined by the number of NP+ (nucleocapsid protein) cells out of the total cells. The data are expressed as mean±SD. n=6 biological replicates. ****, p<0.0001. Altogether, these results demonstrate that ACE2plusC3 cells are highly susceptible to SARS-COV-2 infection.

### 3.2. Characterization of the ACE2plus and ACE2plusC3 Cell Lines

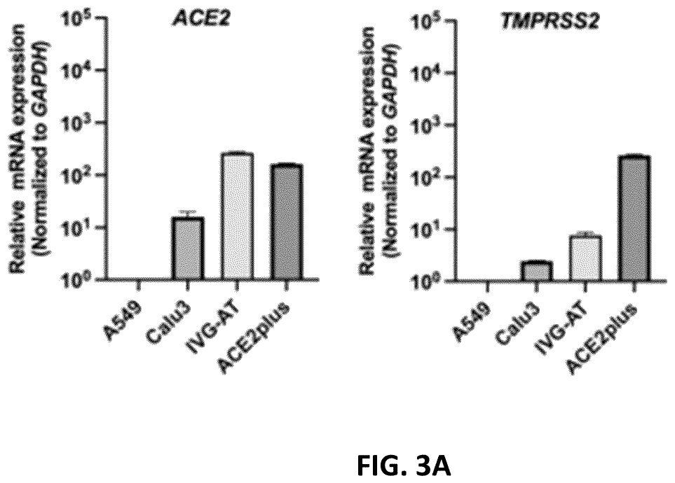

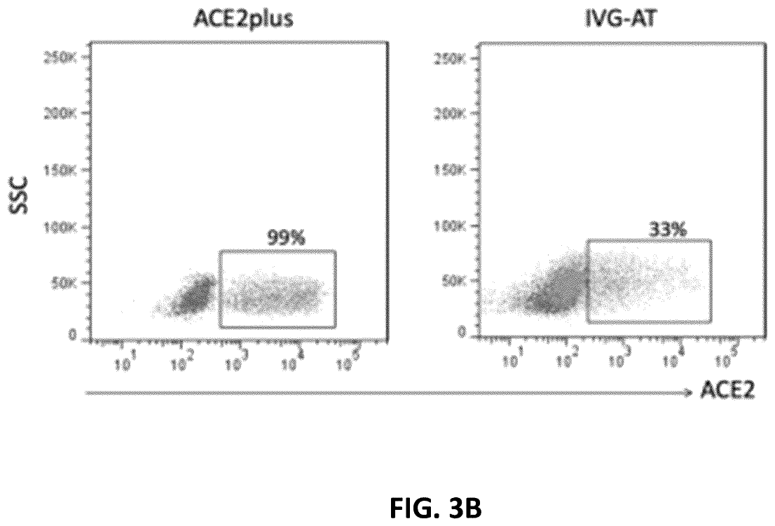

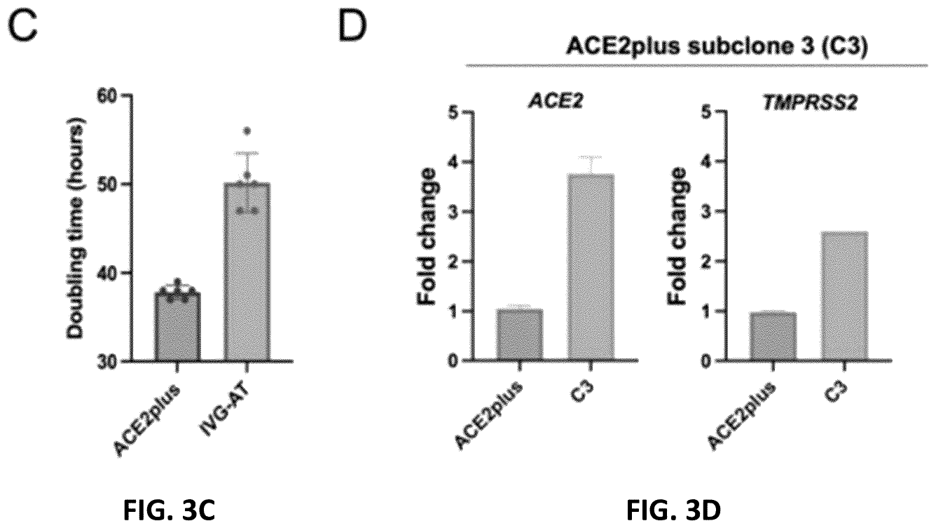

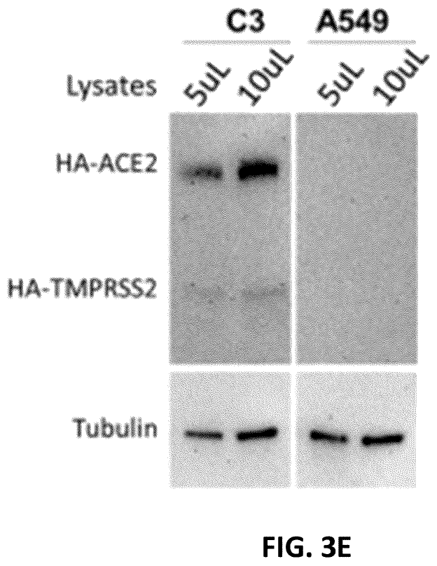

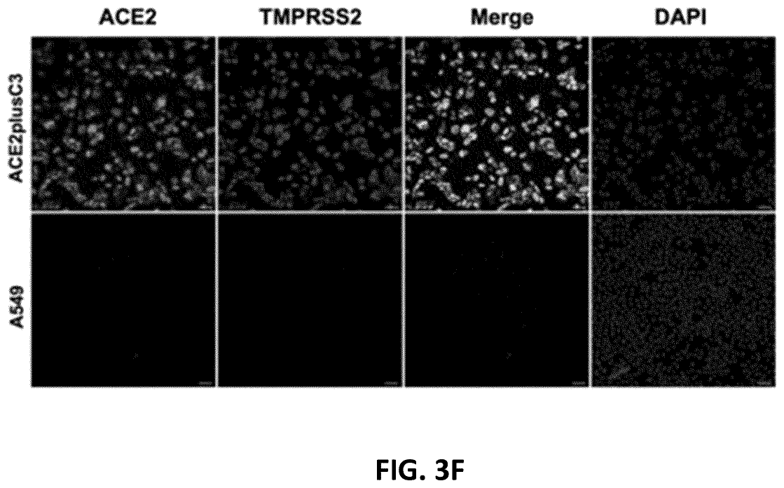

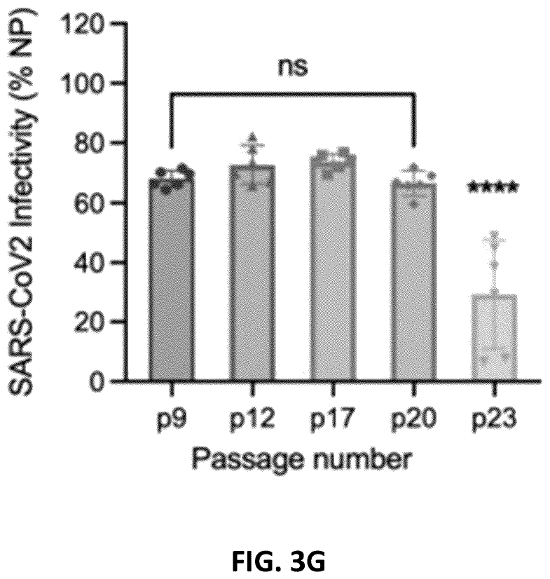

The characterization of the ACE2plus and ACE2plusC3 cell lines. ACE2 and TMPRSS2 are key receptors for SARS-COV-2 entry. To measure levels of ACE2 and TMPRSS2 mRNA expression, RT-qPCR was performed on cell lysates collected from ACE2plus, ACE2plusC3, parental A549, Calu-3, and two commercial cell lines-A549-hACE2 (“IVG-A”, lot 42-01) and A549-hACE2-TMPRSS2 (“IVG-AT”, lot 42-02). As shown in FIGS. 3A and 3D, the mRNA expression levels of ACE2 and TMPRSS2 in the indicated cell lines were measured with RT-qPCR. IVG-A and IVG-A/T commercial cell lines were used as comparators. As expected, both ACE2 and TMPRSS2 mRNA expression levels in the parental A549 cells were negligible. ACE2 mRNA expression levels in ACE2plus and IVG-AT cells were similar. However, ACE2plus cells expressed a higher level of TMPRSS2 mRNA than IVG-AT and Calu-3 lines.

Next, we conducted flow cytometry to determine the cell-surface ACE2 protein expression. The cell surface ACE2 expression level was measured with flow cytometry using live cells. AS shown in FIG. 3B, over 95% of ACE2plus cells were ACE2-positive, whereas IVG-AT cells were only 33% ACE2-positive. The cell-doubling time was determined using a cell growth curve and Celigo Image software. As shown in FIG. 3C, IVG-AT cells grow slowly, with doubling times of approximately 50 hours. In contrast, the growth rate of ACE2plus cells is faster and more consistent than the commercial cell line.

The protein expression levels of ACE2 and TMPRSS2 in cells were examined by immunoblotting and immunofluorescence staining. Scale bar, 100 μm. The virus infectivity remains stable between early and late passages (p9, p12, p17, p20, and p23). ACE2plusC3 cells were infected with WT SARS-COV-2 at an MOI of 0.1 for 48 h. Data are expressed as mean±SD. n=6 biological replicates. ns, not significant; ****, p<0.0001.

Then examined were ACE2 and TMPRSS2 expression in the ACE2plusC3 cell line. As shown in FIG. 3D, these cells expressed higher mRNA levels of ACE2 and TMPRSS2 than the parental ACE2plus cells. FIGS. 3E-F show confirmation of this finding using co-immunofluorescence staining and immunoblotting. A549 cells were included as a control. In FIG. 3G, as expected, the ACE2plusC3 cells showed strong and ubiquitous expression for both proteins, which may contribute to the stable virus infectivity between the early and late passages.

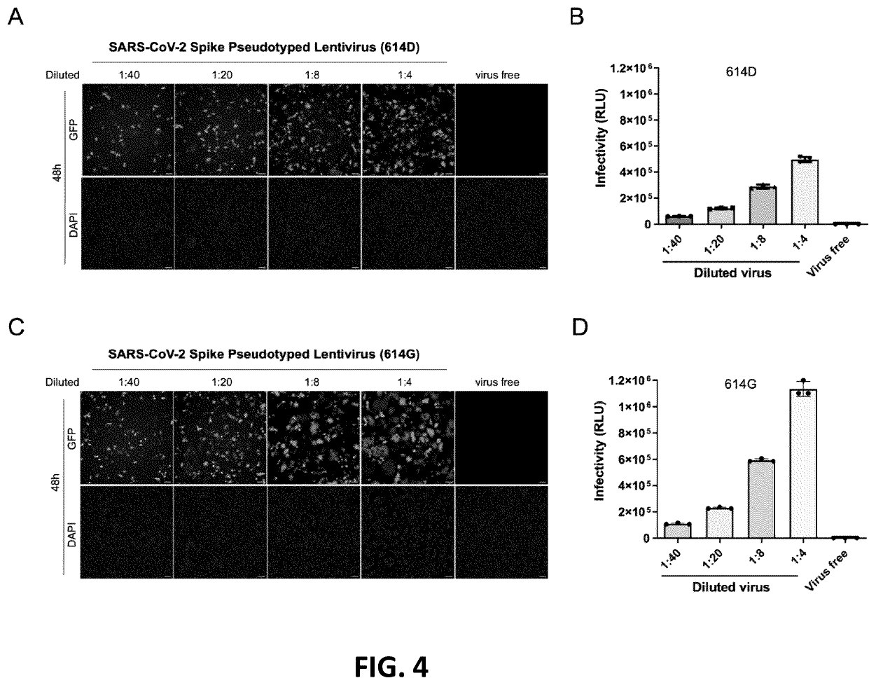

According to recent reports, the spike D614G mutation is associated with ACE2 receptor binding and virus entry. To evaluate this, we used the 614D and 614G strains of S-pseudotyped lentiviral particles to infect ACE2plusC3 cells. The spike 614G virus showed stronger infectivity than 614D in a dose-dependent manner. ACE2plusC3 cells can be infected with SARS-COV-2 Spike-pseudotyped lentivirus (PV). As shown in FIGS. 4A and 4C, representative images of ZsGreen expression in ACE2plusC3 cells at 48 hours post-incubation with a series of diluted 614D or 614G PV. In FIGS. 4B and 4D, infectivity was measured via relative luciferase units (RLU). Each data represents the mean and standard deviation. 100 μM scale bar.

These data support the use of ACE2plusC3 for monitoring SARS-COV-2 pseudotyped lentiviral infection. Our data are also in line with those reported by Cheng et al. (2021), showing that D614G substitution increased the virus titer. Taken together, these results provide evidence that the ACE2plusC3 cell line is a suitable model for SARS-COV-2 research.

### 3.3. Determining if ACE2plusC3 Cells can be Used to Test the Efficacy of Antiviral Drug Candidates Against WT SARS-COV-2 Infection

To evaluate the utility of the ACE2plusC3 model in antiviral drug screening, we used these cells to compare the antiviral efficacy of nine previously identified or potential antiviral drug candidates against SARS-COV-2 infection. Nirmatrelvir, EIDD-1931, and remdesivir have been approved by the FDA to treat COVID-19. Nirmatrelvir (PF-07321332) is an orally bioavailable viral 3C-like protease inhibitor. EIDD-1931, the active metabolite of EIDD-2801, is an orally bioavailable drug that targets viral RNA-dependent RNA polymerase. Nelfinavir, a leading HIV protease inhibitor, has been shown to target the SARS-COV-2 main protease to inhibit virus replication. Camostat mesylate, naphthofluorescein, E64d, and decanoyl-RVKR-CMK have been widely used to study the virus entry and spike processing by targeting host cell proteases. Fluvoxamine is a selective serotonin reuptake inhibitor that is approved by the FDA to treat obsessive compulsive disorder. This drug binds to the sigma-1 receptor on immune cells to reduce inflammatory cytokine production and is being evaluated for the treatment of COVID-19 in randomized controlled trials in humans.

To determine the dose-response curves (DRC) of the antivirals, we inoculated ACE2plusC3 cells with WT SARS-COV-2 for 1 h and incubated them with each drug candidate for 48 h before fixation. Viral nucleocapsid protein (NP) was stained using immunofluorescence, and cell nuclei were stained with DAPI; fluorescence was quantified to determine the inhibition efficacy using the IXM image system and software. In FIG. 4, the DRC analysis of the reference drugs (i.e., EIDD-1931, remdesivir, and nirmatrelvir) showed strong inhibition efficacy against SARS-COV-2-infected ACE2plusC3 cells. Nelfinavir also showed a potent and dose-dependent inhibition of infection, but we noted increased toxicity with ≥25 μM. Cells were treated with camostat mesylate, naphthofluorescein, E64d, or fluvoxamine. Only camostat mesylate treatment significantly inhibited SARS-COV-2 infection without causing cell toxicity. This is consistent with recent reports that camostat mesylate significantly reduces SARS-COV-2-driven entry and infection in primary human lung cells.

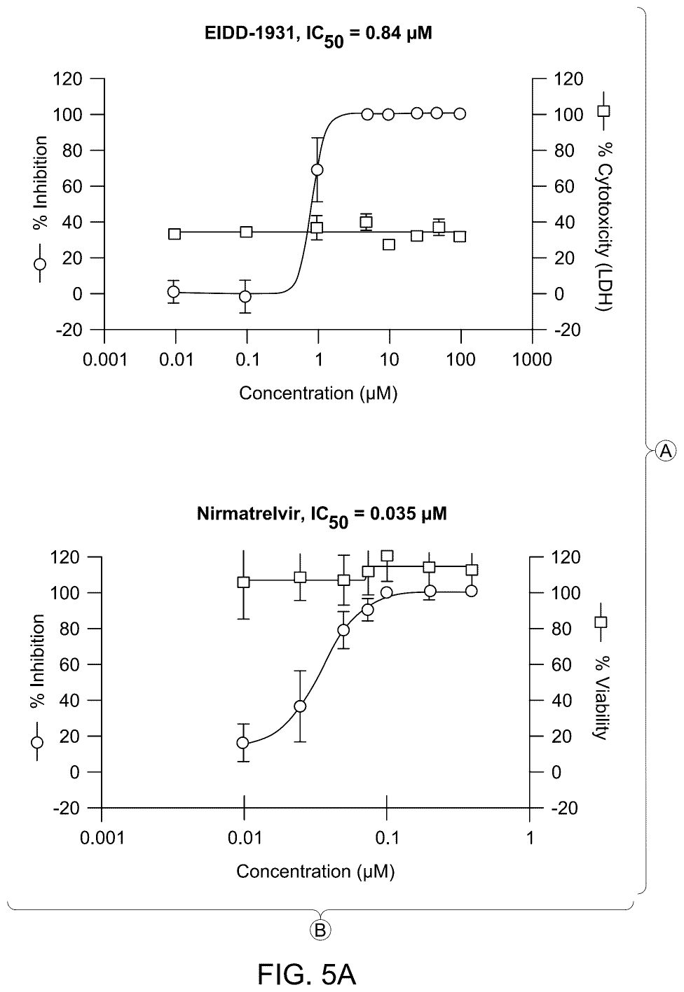

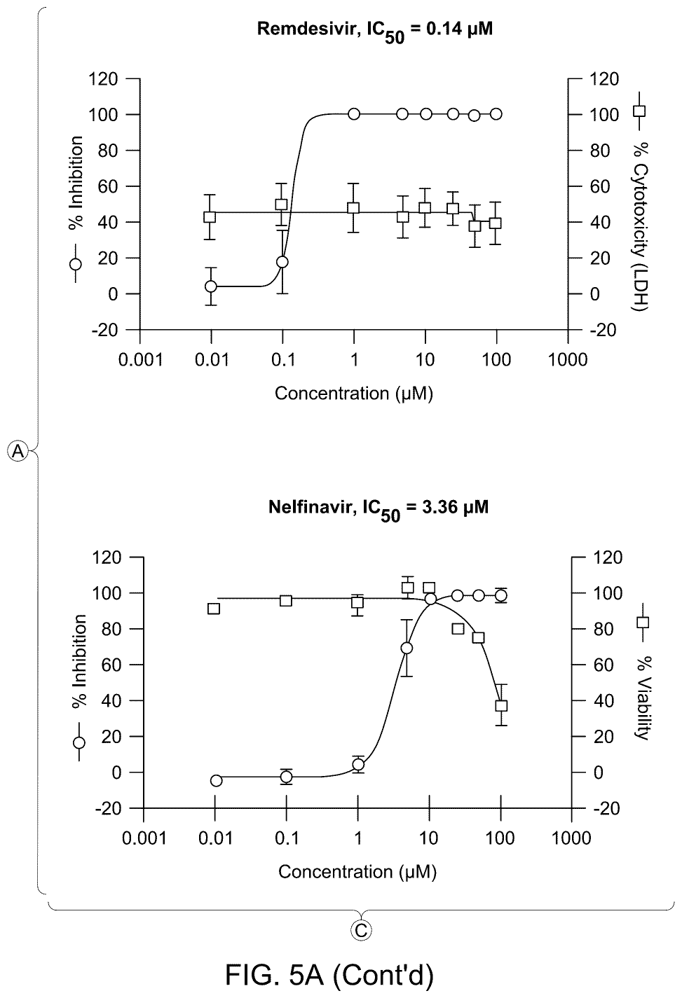

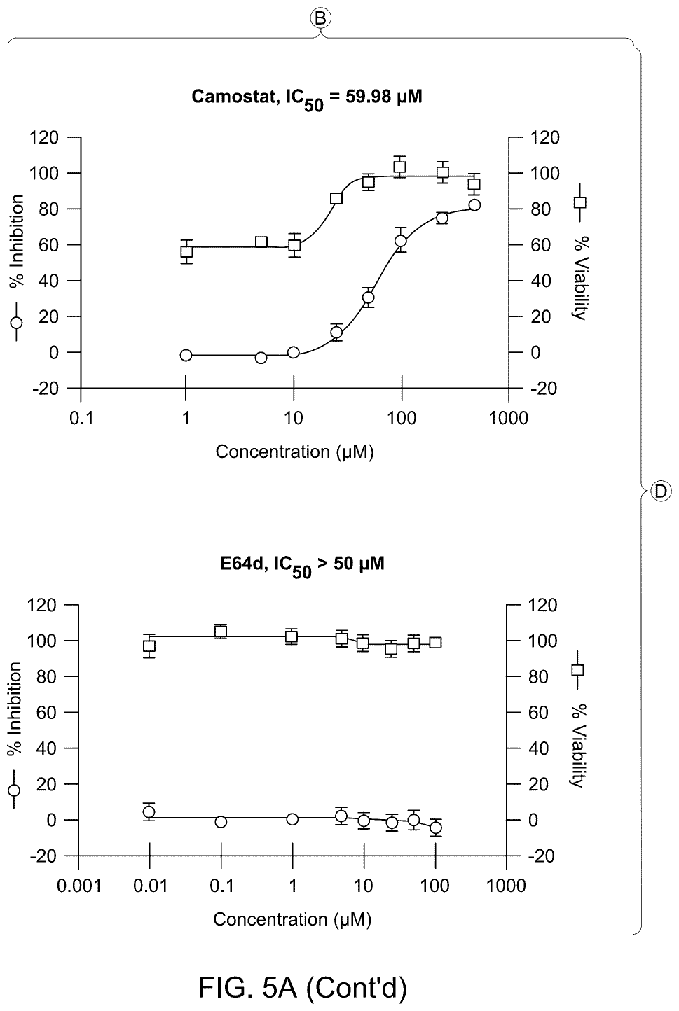

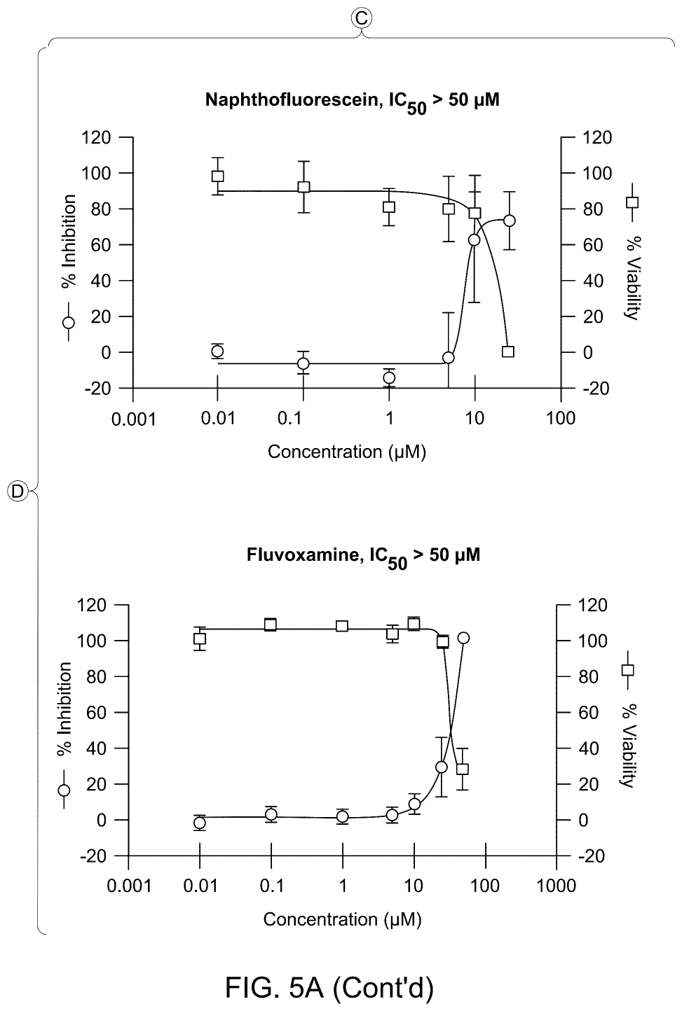

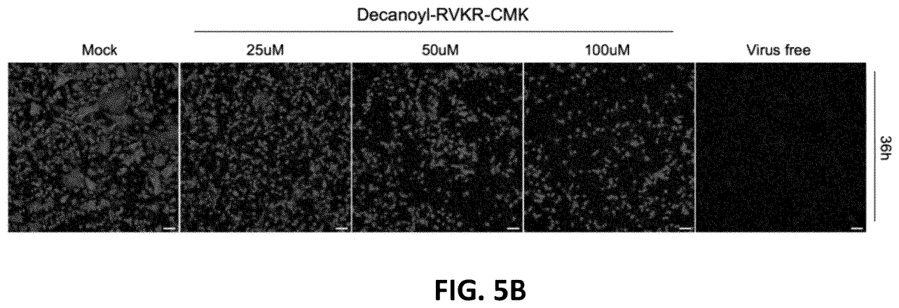

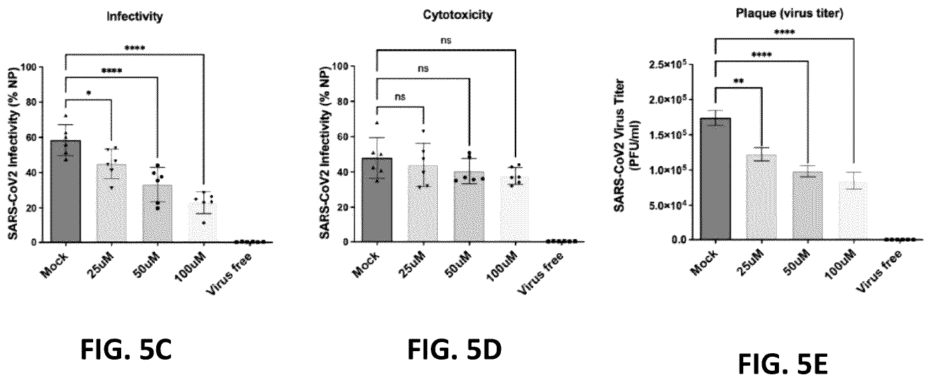

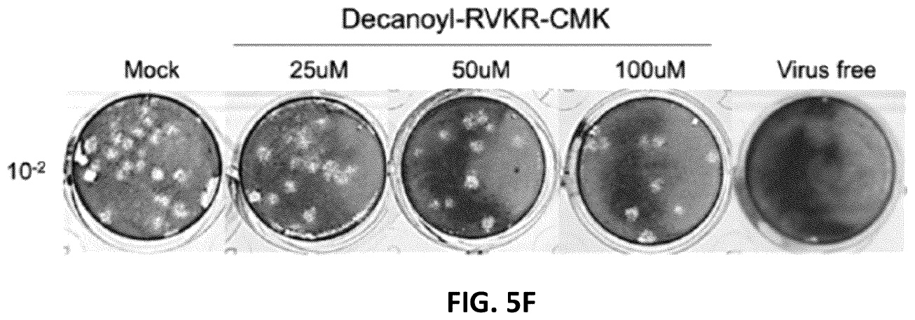

Since several published studies have reported that the cleavage of the SARS-CoV-2 S protein at a putative furin cleavage site (RRARS) at R685/S686 is critical for virus entry, we investigated the efficacy of the furin inhibitor decanoyl-RVKR-CMK and observed a moderate inhibition of infection in the WT SARS-COV-2-infected cells as determined by immunofluorescence and plaque assays. In FIGS. 5B-F, the furin inhibitor decanoyl-RVKR-CMK moderately inhibits SARS-COV-2 infection in ACE2plusC3 cells. Shown in FIG. 4B, microscope images show viral nucleocapsid protein (NP) expression (red) in infected cells, with DAPI (blue) used for nuclear counterstaining. Cells were infected with SARS-COV-2 (WA1/2020) at an MOI of 0.1 in the presence of furin inhibitor for 36 hours. Images were scanned by ImageXpress using 10× magnification. As shown in FIG. 5C, quantifying the virus infectivity via ImageXpress. Shown in FIG. 5D, cytotoxicity was measured by the LDH assay. In FIGS. 5E-F, viral titer quantification by plaque assay of supernatants collected at 36 hours post-infection and example images are shown. Data are representative of the mean and SEM of two independent experiments. ns, not significant; *, p<0.05; **, p<0.01; ****, p<0.0001. These results demonstrate that our ACE2plucC3 cell model can be used to evaluate antiviral drugs and potentially developed for high-throughput screening.

### 3.4. Assessing the Utility of ACE2plusC3 Cells to Identify Potent Antivirals Against Emerging Variants of SARS-COV-2

Given that the emergence of SARS-COV-2 variants with increased transmissibility continues to threaten global public health, evaluated was the efficacy of a panel of antivirals at inhibiting the delta and omicron variants of SARS-COV-2 in our ACE2plusC3 cells. The antiviral activity of EIDD-1931, remdesivir, nirmatrelvir, and nelfinavir against wild-type, delta, and omicron SARS-COV-2 infection in ACE2plusC3 cells. Small molecules were evaluated in ACE2plusC3 cells at the indicated concentrations (μM) or with DMSO control. Cells were infected with wild-type SARS-COV-2 and the indicated variants at an MOI of 0.1 for 48 h. Virus-infected cells were visualized with immunofluorescence staining of SARS-COV-2 NP. Infectivity was measured as described in FIG. 1. Each concentration was performed in sextuplicate with averages and standard deviations indicated. ns, not significant; ***, p<0.001; ****, p<0.0001.

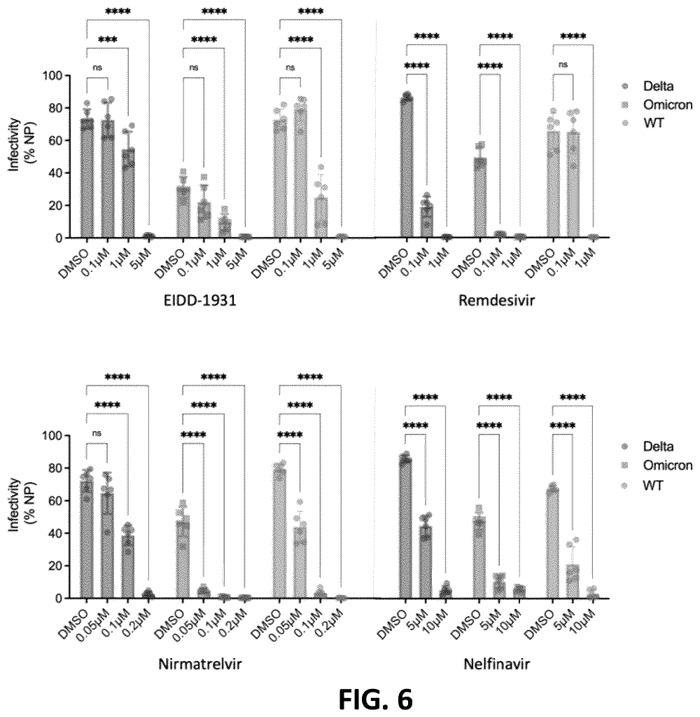

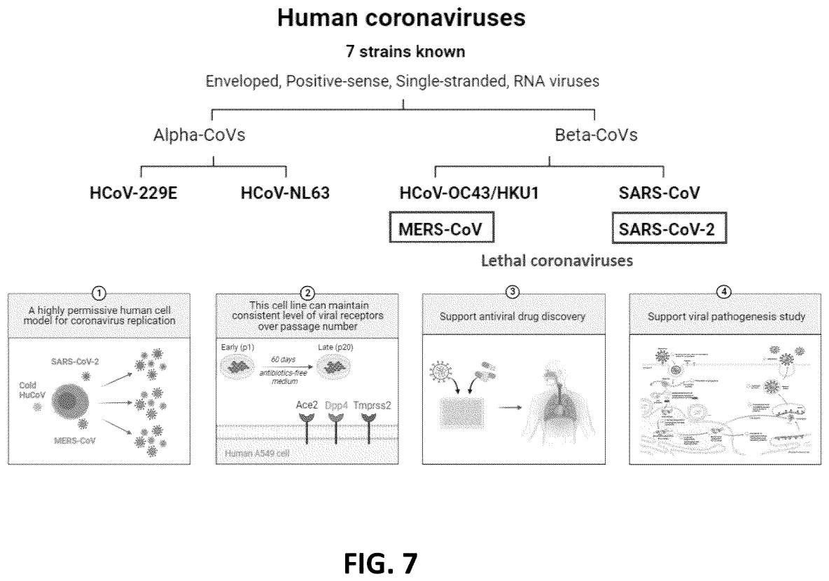

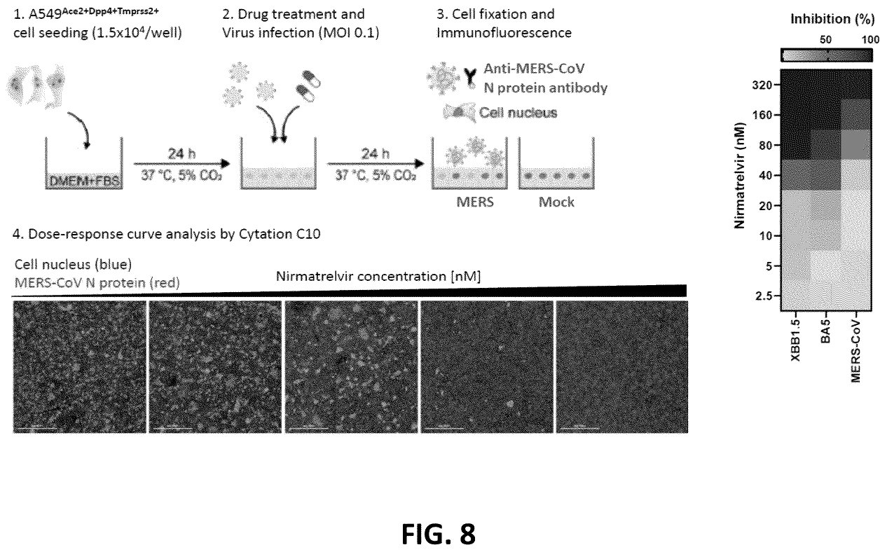

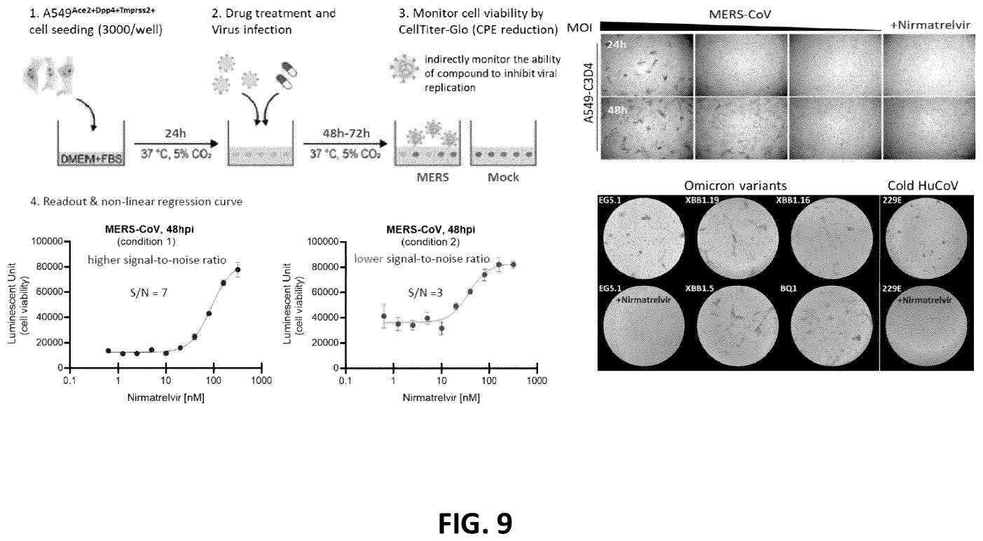

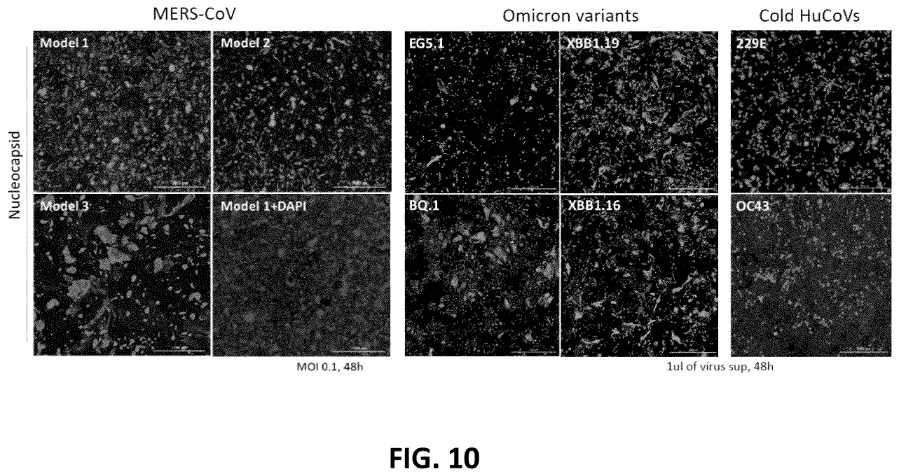

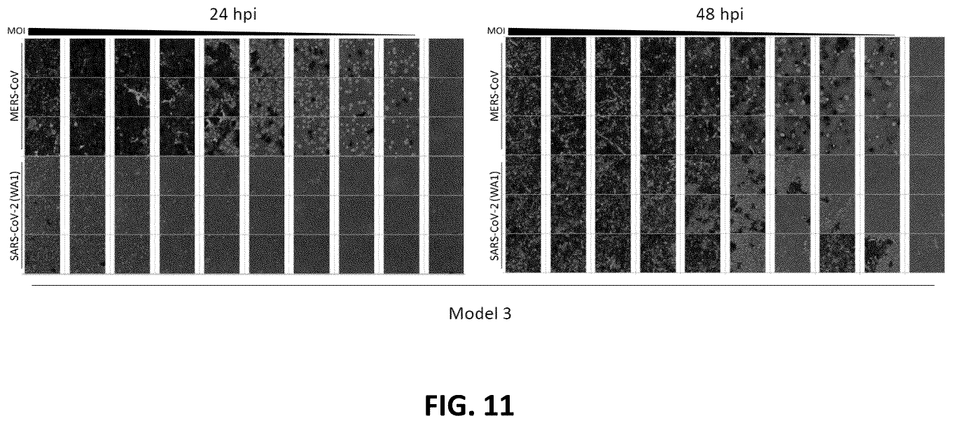

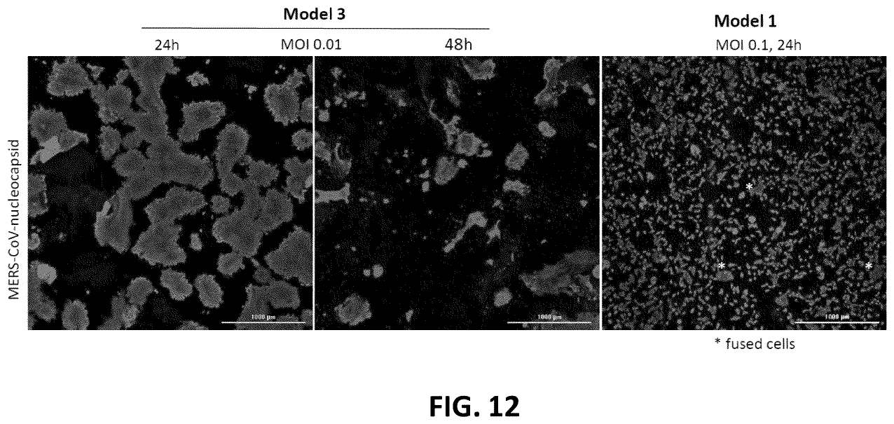

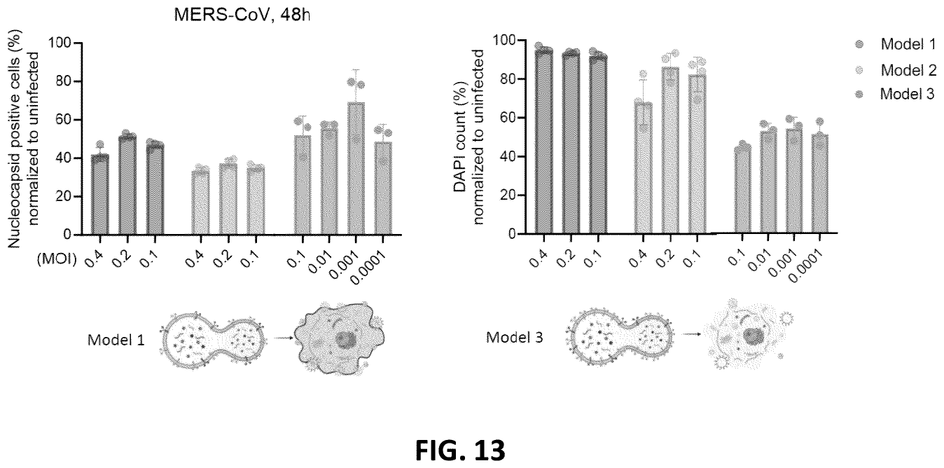

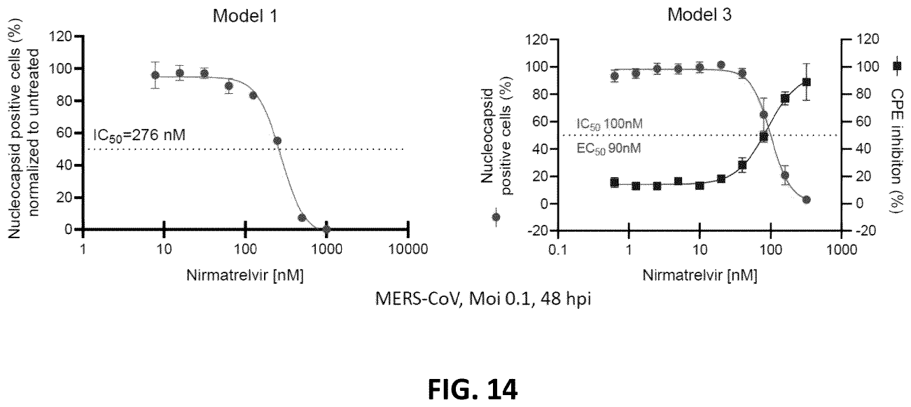

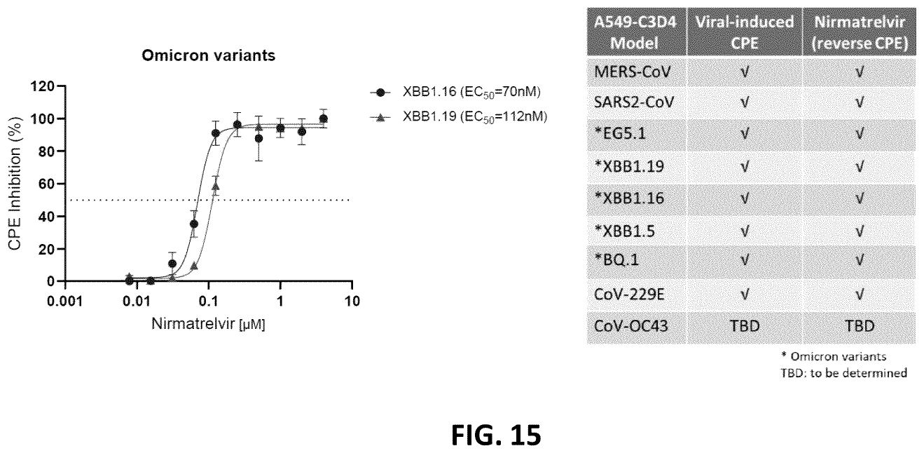

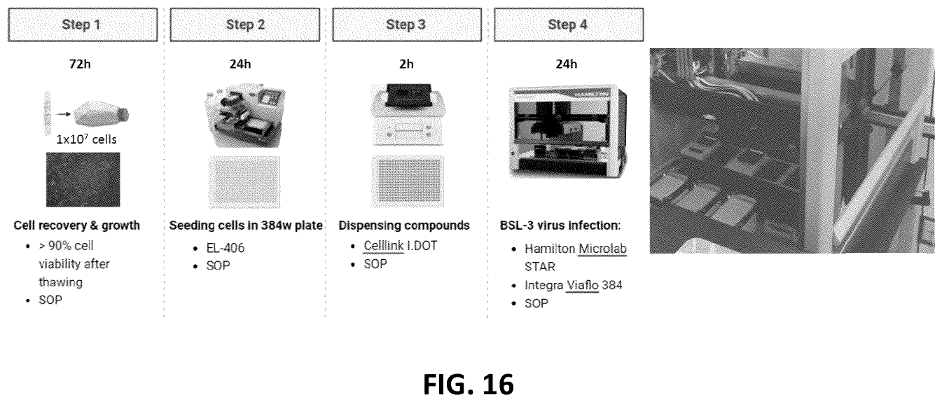

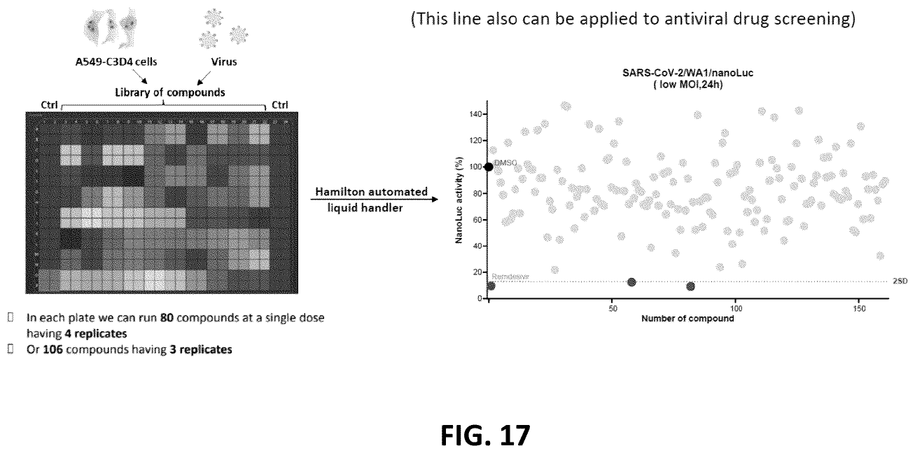

We found that EIDD-1931, remdesivir, nirmatrelvir, and nelfinavir exhibit a dose-dependent inhibition of infection. EIDD-1931 reduces WT, delta, and omicron SARS-CoV-2 infection by at least 50% at 1 μM (FIG. 6 top), with complete inhibition at 5 μM. Remdesivir inhibited the WT virus and the two variants at 1 μM. An exceptionally low concentration of nirmatrelvir (0.1 μM) reduced delta virus infection by 50% and nearly completely inhibited WT and omicron SARS-COV-2 infection. By contrast, nelfinavir did not completely inhibit infection at a higher concentration of 10 μM (FIG. 6 bottom). Compared with WT and delta SARS-COV-2, the omicron variant appears more sensitive to the drug treatment in this model.

### 4. Discussion

The world is now in the third year of the SARS-COV-2 pandemic and facing new challenges from emerging variants. While novel vaccines provide protection and slow the spread of infection, this approach is not feasible in some immunocompromised individuals, and breakthrough infections in vaccinated and boosted individuals have been reported. Antiviral strategies are a promising alternative; thus, our aim was to establish a reliable cell culture system for accelerating the discovery of novel antiviral drugs against SARS-COV-2 and its variants. We generated the ACE2plusC3 cell line that allows researchers to quantitate SARS-CoV-2 infectivity through high-throughput screening of small molecules. Common assays using the pseudotyped virus for antiviral research can be easily transferred to the ACE2plusC3 setting with comparable results.

Several cell culture models are available for monitoring SARS-COV-2 infection and pseudotyped virus activity. Although 293T-ACE2 and Vero E6 have been widely used to study SARS-COV-2 entry, replication, and antiviral treatments, these cells are unsuitable for investigating the pathological mechanisms of the host cell's response to virus infection as they are not derived from human lung tissue, cannot be used to assess cytopathic effects, and do not express type I interferon genes. Calu-3 cells derived from human lung tissue are an alternative system; however, these cells grow slowly and their ACE2 surface abundance is heterogeneous. Unlike Calu-3 cells, A549 cells grow faster and are easier to manage in cell culture. Here, we compared the properties of the commercialized A549-hACE2/TMPRSS2 line (“IVG-AT”) with single-cell-derived ACE2plusC3 cells and found that the ACE2plusC3 cells offer significant advantages in terms of cell proliferation and SARS-COV-2 susceptibility. In addition, ACE2plusC3 cells can be subjected to more antibiotic selection than IVG-AT to establish required stable cell lines. Another advantage is that ACE2plusC3 cells are a more homogeneous cell population than either the parental ACE2plus cell line or the commercial IVG-AT cell line. Our cell model also displayed more sensitive responses to antiviral treatment when compared with commonly used Vero E6 and Calu-3 cell lines, demonstrating that ACE2plusC3 cells are a valuable system for assessing antivirals.

Antivirals can target multiple steps in the SARS-COV-2 entry process, including virus attachment to the cell surface, receptor engagement, protease processing, and membrane fusion. The entry process mainly relies on the receptor-binding domain (RBD) and S2 domain in the SARS-COV-2 spike protein. Viral entry also depends on host factors. In addition to ACE2, several molecules have been suggested to serve as alternative receptors for SARS-COV and SARS-COV-2 entry, including C-type lectins, DC-SIGN, and L-SIGN. These receptors bind a wide range of viruses by recognizing the glycans on the virion surface and promote viral entry by allowing the virus to attach to the target cell, acting as attachment factors for virus entry. Although those receptors increase virus entry, they do not support virus infection in the absence of the ACE2 receptor. In this work, we show that ACE2pluC3 cells were susceptible to infection by multiple SARS-COV-2 variants, allowing for studies with a broad range of viral strains. Using ACE2plusC3 cells, researchers can now examine additional attachment factors and their contributions to susceptibility to virus infection.

The results demonstrate the potential of ACE2plusC3 cells for examining SARS-COV-2 variants, particularly the omicron variant that has been reported to have unique properties. In our system, the omicron variant demonstrated decreased infectivity compared with the WT virus and the delta variant. This decrease could be due to attenuation. Although we used a low-passage virus stock to mitigate attenuation, further studies are needed to rule it out. In our antiviral drug test, we also found that the omicron variant appears more sensitive to antiviral drug treatment than the WT and delta variant. Recently, Hui et al. reported that omicron (B.1.1.529) replicates faster than other SARS-COV-2 variants in the human bronchi but less efficiently in the lung parenchyma. They found that omicron infection was less dependent on TMPRSS2 activities and could enter cells primarily through the endocytic pathway, while delta preferentially enters cells through cell surface fusion. It is unclear whether omicron is more dependent on the endocytic pathway for entering cells or if the omicron spike protein inefficiently uses the cellular protease TMPRSS2 for virus entry in our cell model. The omicron variant has 30 mutations in the spike protein, half of which are in the RBD, suggesting that this variant may be immunologically resistant to antibody-mediated protection-further emphasizing the need for antivirals. Moreover, the spike protein is just one of the structural proteins. Other viral proteins, like the membrane (M), envelope (E), and nucleocapsid (N) proteins, may also contribute to virus entry and attenuate replication. Interestingly, Gerard Goh et al. suggested that the omicron virion has a harder shell with attenuated replication than others based on their computed data from a shell disorder model. According to their prediction, the outer shell disorder of omicron is lower than that of other variants and might provide the omicron virion with a rigid outer shell that protects the virus from damage by salivary or mucosal antimicrobial enzymes. In a similar strategy, they predicted that the inner shell disorder of the omicron is inherently attenuated, like pangolin-CoV 2017, an attenuated precursor of SARS-COV-2 that might have jumped from pangolins to humans in 2017. Using the ACE2plusC3 system, we can begin to address these questions about the omicron variant and future variants.

In conclusion, developed was, among other things, a suitable human cell model for SARS-COV-2 susceptibility. The data on authentic virus infection, pseudotyped virus infection, and antiviral assays highlight the potential of the ACE2plusC3 cell model for studying emerging SARS-COV-2 variants and antiviral drug screens.

### Additional Testing Material and Methods

### Cell Lines

A549-Ace2-tmprss2 cells were established as previously described (Chang, Parsi et al. 2022). VeroE6-Tmprss2 cells are purchased from Xenotech. 293T and MRC-5 cells were a gift from Dr. Stephen Goff. All cell types were cultured at 37° C. in Dulbecco's Modified Eagle Medium (DMEM) supplemented with 10% heat-inactivated FBS, 100 U/mL penicillin-streptomycin, and 1% L-glutamine.

### Generation and Selection of A549-Ace2-Dpp4-Tmprss2 Cells

The pLEX307-DPP4 plasmid was gifted by Dr. Alejandro Chavez and Sho Ikctani and was used to establish A549-Ace2-Dpp4-Tmprss2 (ADT) cells. Briefly, A549-Ace2-Tmprss2 cells were infected with lentivirus carrying the Dpp4 gene and sorted using an anti-Dpp4 antibody (Biolegend, #302704) at 72 h post infection. Selected clones were cultured in the presence of blasticidin (1 μg/mL), and the medium was changed every two days.

### Viruses and Infection Protocols

The SARS-COV-2 isolate JN.1 (EPI_ISL_18669842) was isolated from a nasopharyngeal sample at the Hackensack Meridian Health Center for Discovery and Innovation (HMH-CDI). Omicron subvariant EG5.1 (NR-59503), MERS-COV (NR-44260), OC43-CoV (NR-56241), and 229E-CoV (NR-52726) were provided by BEI Resources. SARS-CoV-2 and MERS viruses were propagated in VeroE6-Tmprss2 cells in a growth medium consisting of DMEM and 2% heat-inactivated FBS. OC43 and 229E viruses were propagated in MRC-5 cells. Virus infections were performed for 48 h at 37° C. in infection medium (DMEM, 10% FBS). Only OC43-CoV and 229E-CoV were growing at 35° C. All experiments with SARS-COV-2 and MERS-COV were performed in biosafety level 3 laboratories at HMH-CDI. OC43-CoV and 229E-CoV infections were performed in biosafety level 2 laboratories according to standard operating procedures and approved by HMH-CDI Biosafety Committec.

### RNA Isolation and RT-PCR

RNA was isolated from cell lysates using TRIzol reagent and stored at −80° C. for RT-qPCR. The isolated cellular RNA was incubated in gDNA Wipeout Buffer to remove contaminating genomic DNA. The purified RNA was used for cDNA synthesis using the QuantiTect Reverse Transcription Kit (Qiagen, 205311). The resultant cDNA was used to measure the mRNA expression levels of Dpp4, Ace2, and Tmprss2 by qPCR with Applied BiosystemsTM SYBR Green reagent and gene-specific primers. (gene-specific primers Dpp4: sense) ATTCAATATCTCCTGATGGGCAGT [SEQ. 10]; (gene-specific primers Dpp4: antisense) CACTAAGCAGTTCCATCTTCCAC [SEQ. 11]. (Ace2: sense) GGGATCAGAGATCGGAAGAAGAAA [SEQ. 12], (Ace2 antisense) AGGAGGTCTGAACATCATCAGTG [SEQ. 13]. (Tmprss2: sense) AATCGGTGTGTTCGCCTCTAC′ [SEQ. 14]; (Tmprss2: antisense) CGTAGTTCTCGTT CCAGTCGT [SEQ. 15]. GAPDH was used as an endogenous control gene (sense TCCTCCACCTTTGACGCT [SEQ. 16] and antisense TCTTCCTCTTGTGCTCTTGC) [SEQ. 17].

### CPE Reduction Assay

A CPE assay was used to measure antiviral effects against MERS. 20 μL-3000 cells of A549-ADT cells were added to each well of a 384-well tissue culture plate (Corning Inc.) and infected with 20 μL aliquots of SARS-COV-2 or MERS-COV at MOI=0.1 with the indicated compound concentrations. After 48 h of incubation, 40 μL CellTiter Glo (Promega) was added to each well. Following incubation at room temperature for 10 min, the plates were read using a Tecan Infinite M200PRO plate reader to measure the cell viability by luminescence.

### Immunofluorescence Assay

Specific immunofluorescence staining for viral nucleocapsid protein (NP) was performed to determine viral replication activity. Infected cells were fixed with 4% PFA for 30 min at room temperature, gently washed twice with phosphate-buffered solution (PBS), and then permeabilized with 0.1% Triton X-100 in PBS. The cells were then blocked with blocking solution (0.1% Triton X-100 and 0.2% BSA in PBS) for 30 min. NP-specific antibodies (40143-MM08, SARS-COV-2; 40068-MM10, MERS; 40643-T62, OC43; 40640-T62, 229E) were used at 1:1000 dilution and incubated overnight at 4° C. After washing three times with blocking solution, the cells were treated with secondary antibodies conjugated with Alexa-594 (Invitrogen) at 1:2000 dilution and incubated for 1 h at 4° C. Cell nuclei were counterstained with 4′,6-diamidino-2-phenylindole (Abcam) for 15 min at room temperature. Images were acquired with Cytation C10 using immunofluorescence with 4× objective lens. Images were processed using the Gen 5 Image Software (Agilent BioTek).

### Cytotoxic Concentration Assay

The half-maximal cytotoxic concentration (CC50) was assessed in ADT cells in 384 well plates. 20 ul of cells (˜3000) per well were plated. After 24 h, the compounds were added to the wells with indicated concentrations, and the plates were incubated for 72 h. The toxicity of the compounds was measured using 20 ul of CellTiter Glo per well. Following incubation at room temperature for 10 min, the plates were read using a Tecan Infinite M200PRO plate reader to measure the cell viability by luminescence.

### Flow Cytometry Analysis

Approximately one million live cells from each ADT subclone were trypsinized as single cell suspension and stained with anti-Dpp4 antibody conjugated with FITC and anti-Ace2 antibody conjugated with APC simultaneously. Briefly, trypsinized cells were washed twice by PBS and then stained with goat anti-Ace2 antibody (R&D, AF933), mouse anti-Dpp4 antibody (Biolegend, #302704) on ice for 20 min. After washing, cells were analyzed using BD LSR Fortessa (BD Biosciences). Mock cells without adding antibodies were used as a control for gating Ace2+/Dpp4+ cell populations. The fluorescence intensity was measured using BD DIVA software.

### Additional Results

### Generation of the MERS-COV-Susceptible A549-Ace2-Dpp4-Tmprss2 Cell Line

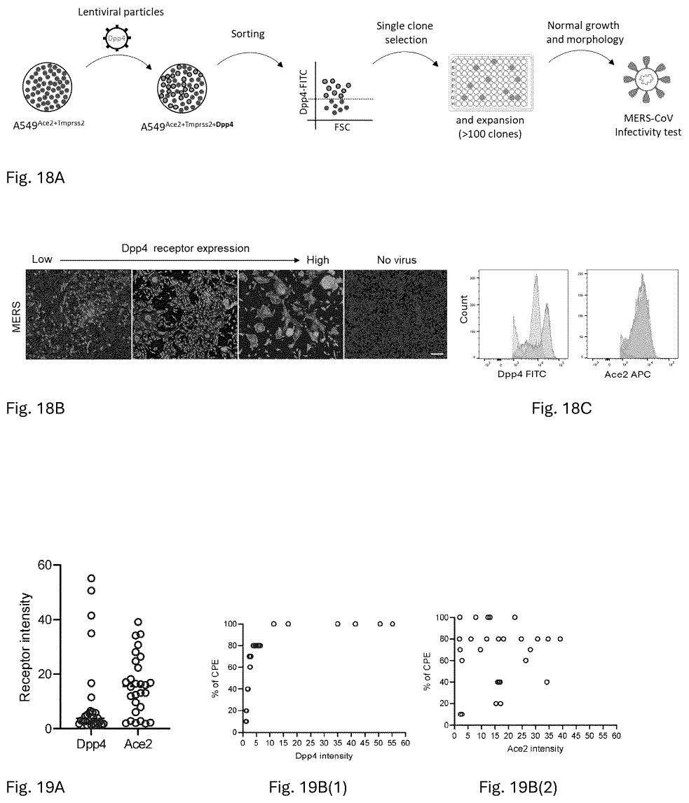

A549-Ace2-Tmprss2 cells were transduced with lentivirus encoding the human Dpp4 gene and blasticidin resistance gene and selected by culturing in the presence of blasticidin. Cells expressing different levels of the Dpp4 receptor were selected by single-cell sorting using Dpp4-specific antibodies (FIG. 18A). Selected cells were expanded and infected with MERS-COV at an MOI of 0.1 for 48 h. The infected cells were fixed and analyzed by immunofluorescence microscopy using antibodies that recognize the viral nucleocapsid protein. We found that Dpp4 expression was correlated with virus-induced CPE and cell death (FIG. 18B). In contrast, the selected cells expressed a similar level of Ace2 and were independent of MERS-COV-induced cell death (FIG. 18C).

### The Susceptibility of the Selected Clone to SARS-COV-2, MERS-COV, OC43, and 229E

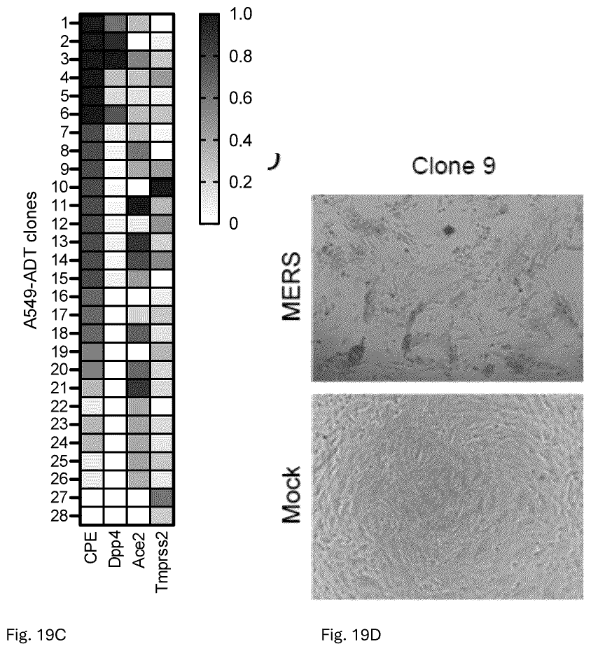

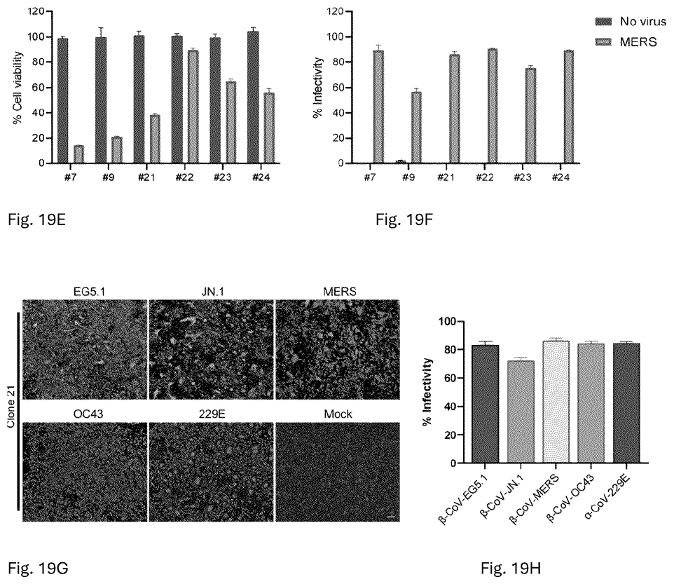

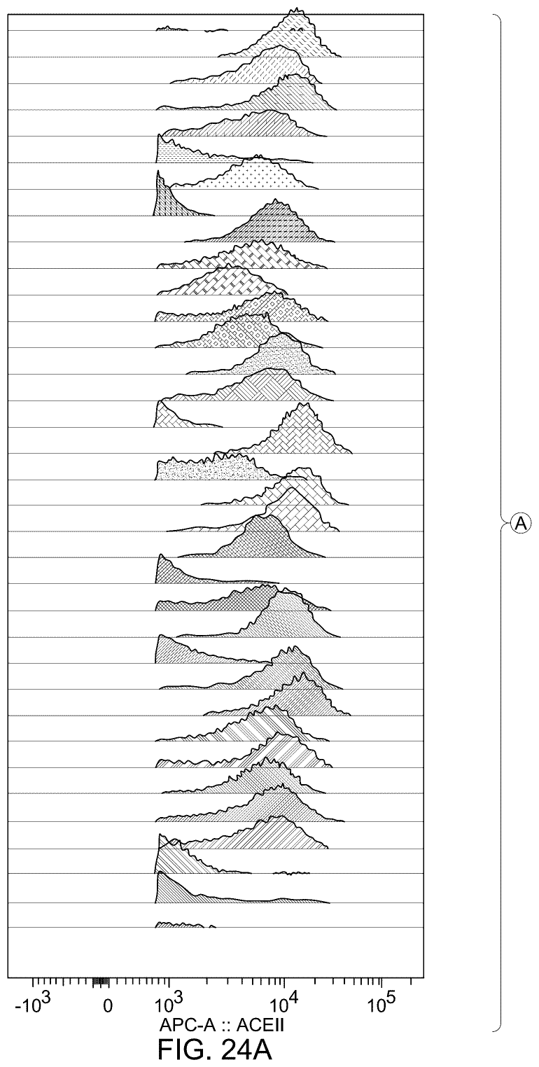

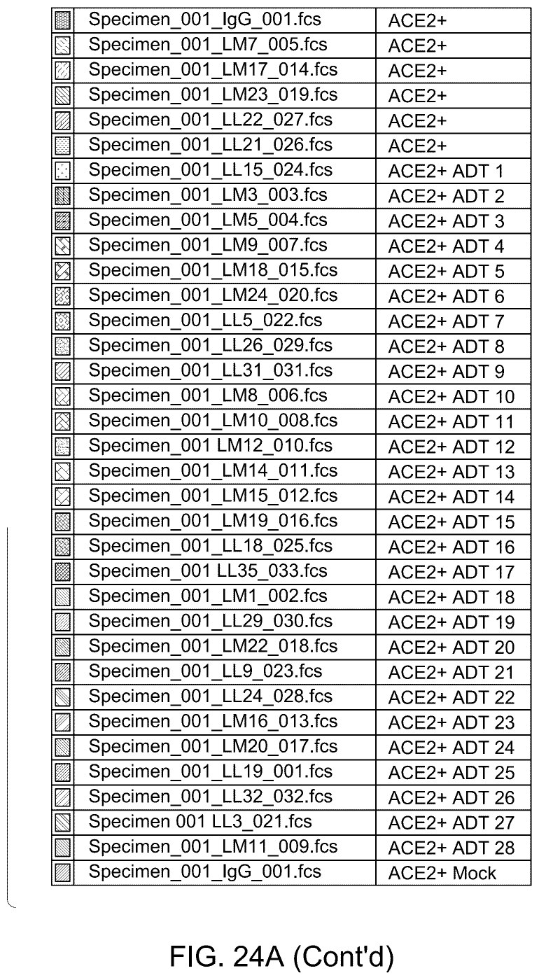

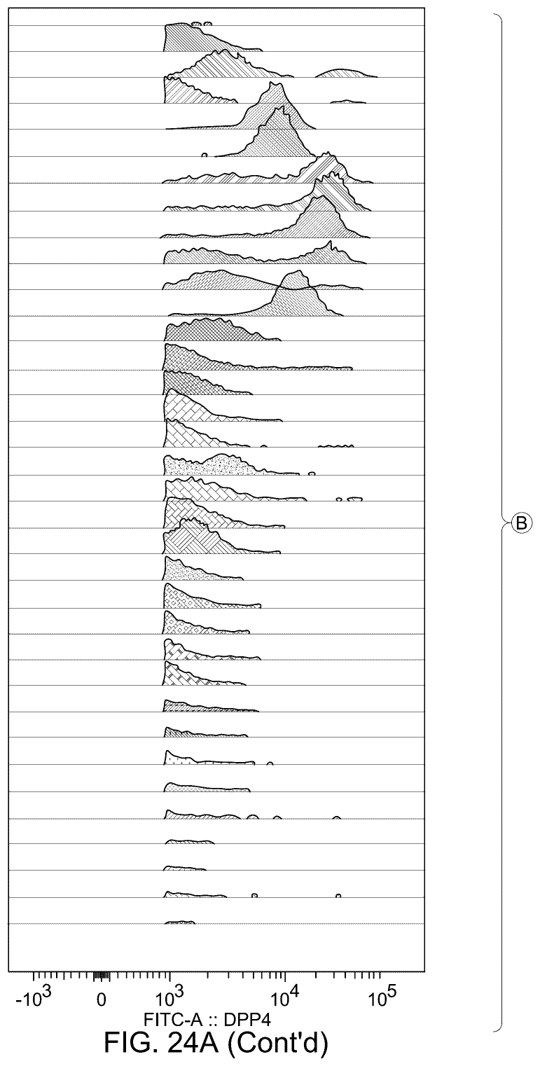

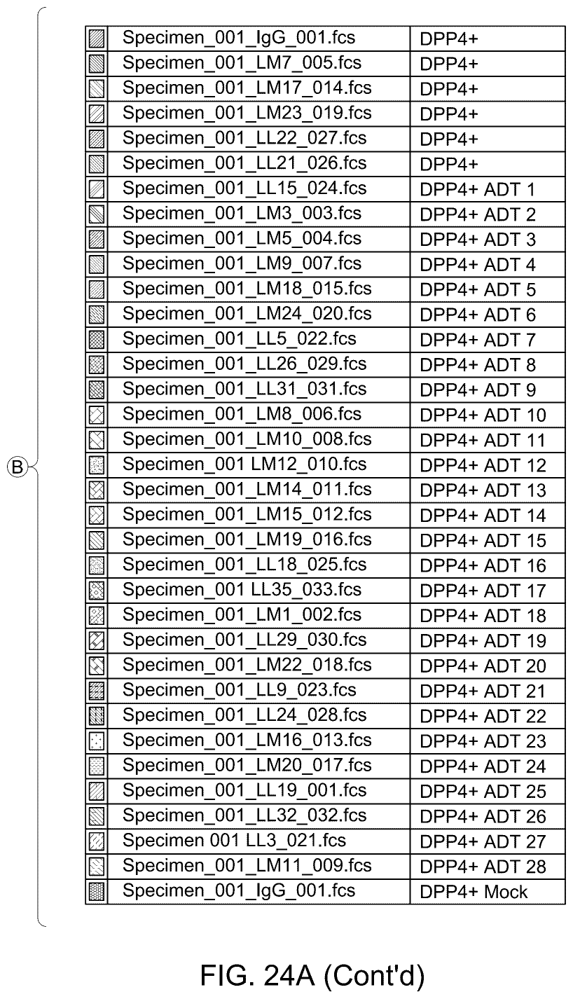

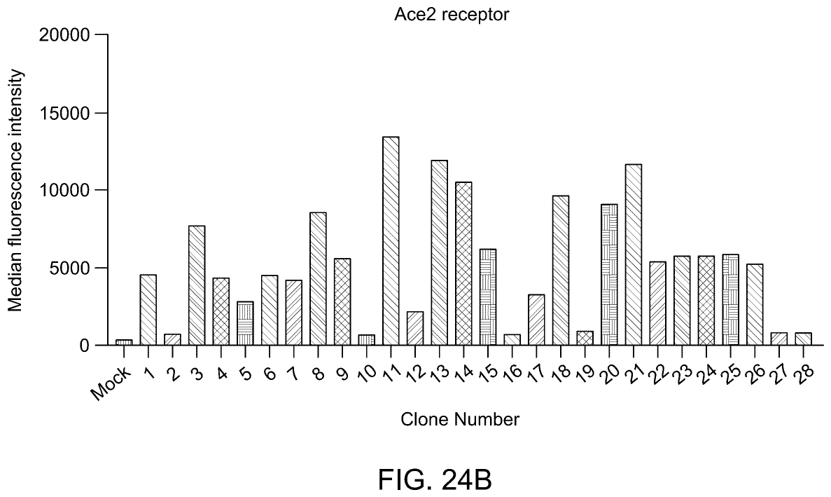

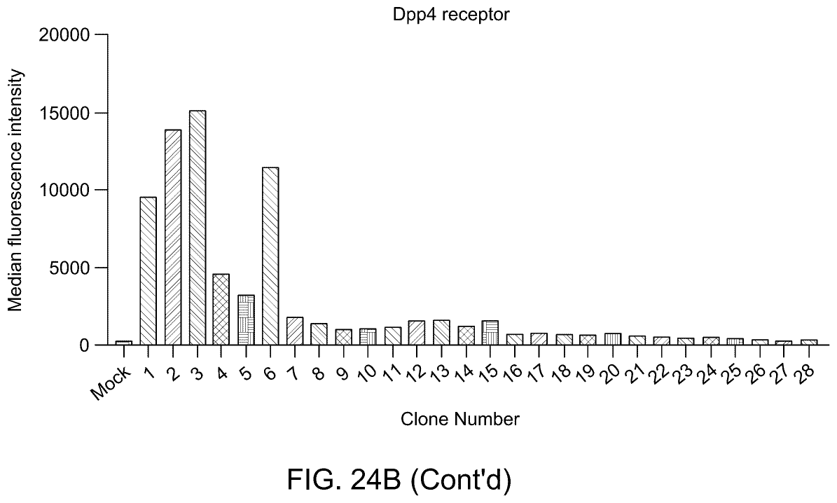

Flow cytometry was used to assess the cell surface expression levels of Ace2 (the SARS-COV-2 receptor) and Dpp4 (the MERS-COV receptor) in sorted clones (FIG. 24A-24B). As illustrated in FIG. 19A, Ace2 and Dpp4 expression levels differed among the A549-ADT clones. A positive relationship was observed between CPE degree and Dpp4 expression levels, whereas MERS-COV-induced CPE was not dependent on Ace2 expression (FIG. 19B(1) and FIG. 19B(2)). Quantitative PCR was also used to assess the Tmprss2 expression levels. The majority of clones exhibited comparable Tmprss2 expression. Among the 28 clones examined, clones 7 and 9 expressed high levels of Dpp4; however, clones 21, 22, 23, and 24 expressed lower levels of CPE and Dpp4 (FIG. 19C). As shown in FIG. 19D, clone 9 exhibited significant cell death following MERS infection, despite its high infectivity rate of approximately 50%. Clones 7 and 9 may not be suitable for immunofluorescence-based antiviral assays because of their low viability after infection (<20% cell viability). In contrast, clones 21, 22, 23, and 24 were suitable for immunofluorescence assays (FIG. 19E). Those results highlight the importance of Dpp4 receptor in MERS-COV infection and suggest that cells with higher Dpp4 expression may be more susceptible to MERS-COV-induced cell death. Because Ace2 expression level in clone 21 was higher than others, we demonstrated its susceptibility to omicron subvariants EG5.1, JN.1, and cold coronaviruses such as OC43-CoV and 229E-CoV. As shown in FIG. 19G and FIG. 19H, clone 21 is highly permissive to omicron subvariants and cold coronaviruses as well. Additionally, we also evaluated its susceptibility to NL63-CoV (data not shown), but the infection rate was not as high as that observed with OC43-CoV and 229E-CoV.

### The Selected Clone is Suitable for Testing Antiviral Compounds

To simplify the cell-based assay and adapt it for antiviral screening, used was the reference compound nirmatrelvir to further test selected clones using imaging and CPE-based assays. Nirmatrelvir (PF-07321332) is an orally bioavailable viral 3C-like protease inhibitor and has been approved by the FDA to treat COVID-19 (Hashemian, Sheida et al. 2023).

As illustrated in FIG. 20A, comparable dose-dependent curves and IC50 values were observed for clones 7, 21, 23, and 24. Clone 22 did not exhibit a sigmoidal curve when using the CPE-based detection method. However, clone 21 demonstrated an IC50 value similar to VeroE6-Tmprss2 cells with the treatment of P-gp inhibitor in the image-based readout (FIG. 19B(1) and FIG. 19B(2)). Furthermore, clone 21 exhibited morphological characteristics similar to the parent A549 cells and demonstrated the highest Ace2 expression among the clones. Based on these factors, clone 21 was chosen for the subsequent experiments presented in the study.

### Comparison of the Broad-Spectrum Activity of Three 3CL-Protease Inhibitors

To demonstrate the application of A549-ADT21 in assessing broad-spectrum antiviral activity, we examined the efficacy and potency of three SARS-COV-2 3CLpro inhibitors: niamatrelvir (PF-37321332), pomotrelvir (PBI-0451) and ensitrelvir (S-217622) (Hashemian, Sheida et al. 2023, Sasaki, Tabata et al. 2023, Tong, Keung et al. 2023).

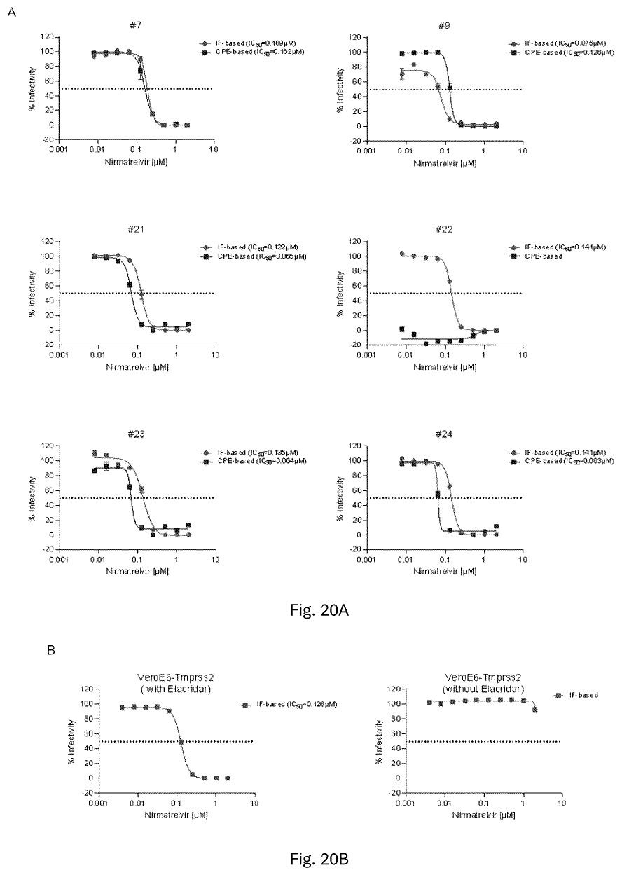

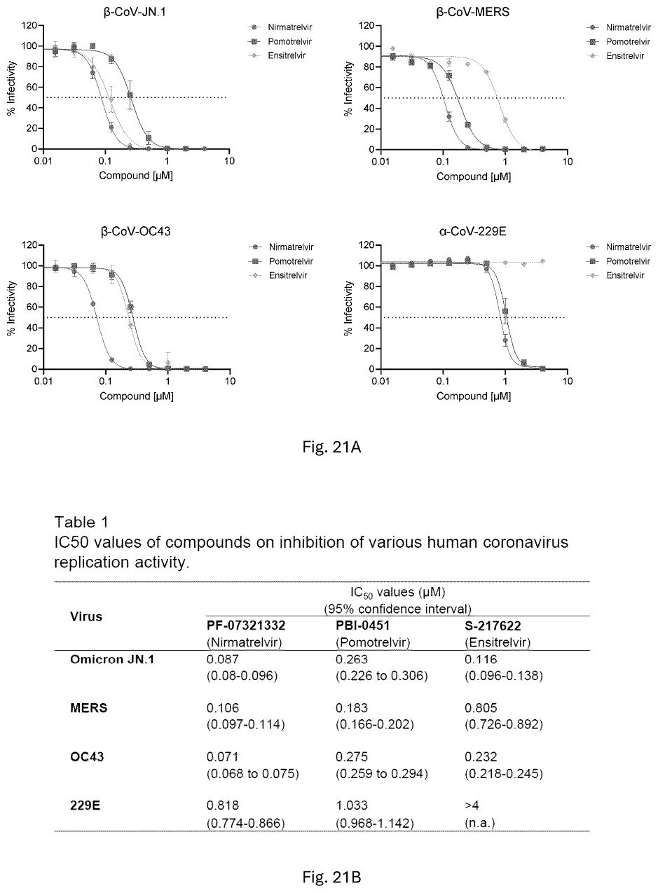

Then compared were the above inhibitors activity against a range of human coronaviruses, including omicron JN.1, MERS-COV, OC43-CoV, and 229E-CoV. In our cell-based assays, the three compounds demonstrated significant efficacy against omicron JN.1, MERS-COV, and OC43-CoV, with IC50 values ranging from 0.087 to 0.805 μM. However, ensitrelvir showed no inhibitory effect on cells infected with 229E-CoV (FIG. 21B). On the other hand, niamatrelvir and pomotrelvir successfully inhibit the 3CLpro of 229E-CoV, although their potency is not as strong as their activity against beta-coronavirus, both compounds exhibited efficacy against alpha-coronavirus, with IC50 values ranging from 0.818 to 1.033 μM (FIG. 21B Table 1). Those results suggest that ensitrelvir may possess a more limited spectrum of activity compared to niamatrelvir and pomotrelvir. Further studies on structure-active-relationships for different coronaviruses may be required.

### Antiviral Activity of Halofantrine Against SARS-COV-2 Infection

The ongoing emergence of new SARS-COV2 strains at a rapid pace underscores the need for developing new antivirals at an equally rapid pace. Developing new antivirals from scratch is challenging. Therefore, repurposing existing drugs has become a promising strategy for rapid identification of potential treatments (Sarhan, Ashour et al. 2021). The 13 repurposed drugs, which are already used for malaria treatment, were generously provided by Medicines for Malaria Venture (MMV) and employed for screening against SARS-COV2 omicron JN. 1 and MERS-COV using the A549-ADT cell model.

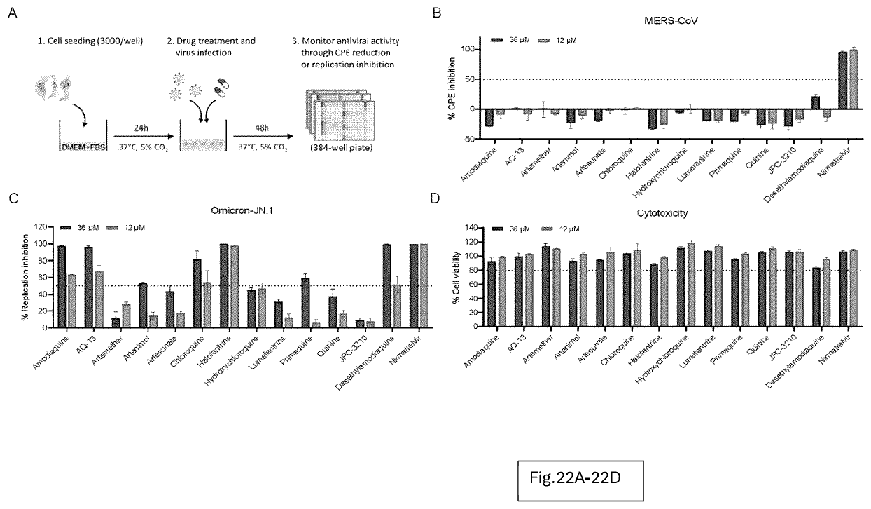

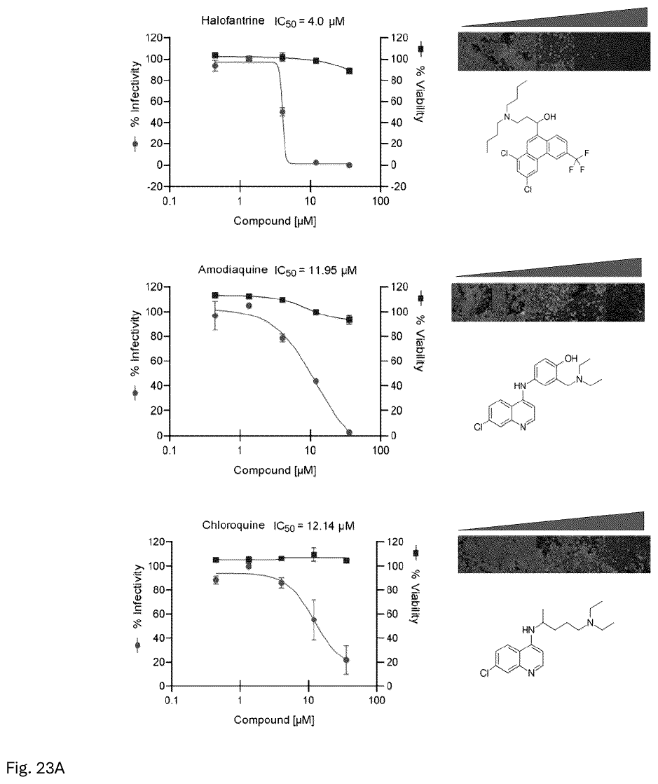

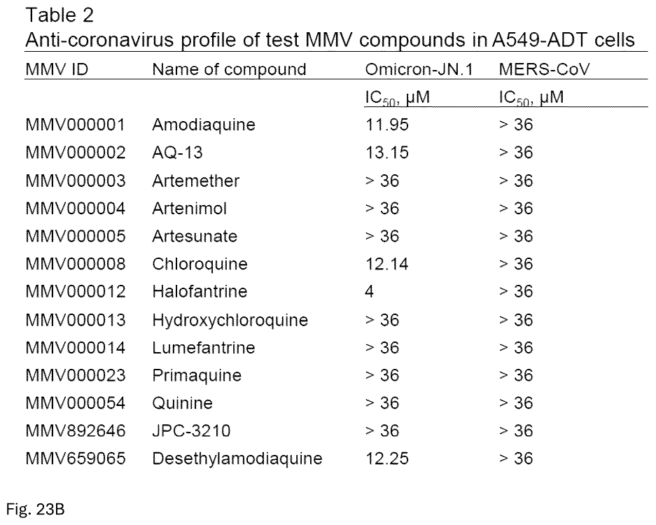

To evaluate its potential for antiviral screening, cells were seeded in 384 well plate at a density of 3000 cells/well in 20 ul of complete DMEM medium. The plates were incubated for 24 h at 37° C. in a humidified CO2 incubator. The following day, the compounds were added to the wells in duplicate at 36 μM and 12 μM concentrations, followed by infection with MERS-COV and JN.1 at MOI 0.1 for antiviral screening (FIG. 22A). After 48h incubation, the MERS-COV-infected plates were analyzed for CPE reduction using CellTiter Glo reagent to determine cell viability by the quantification of ATP. None of the 13 compounds showed the CPE inhibition (FIG. 22B). Nirmatrelvir, used as a control, showed approximately 90% CPE inhibition.

In parallel, the JN. 1 infected plates were analyzed for replication inhibition by staining viral nucleocapsid protein. Some compounds, such as amodiaquine, chloroquine, and halofantrine showed approximately 80-95% replication inhibition at a concentration of 36 μM (FIG. 22C). For the cytotoxicity assay, cells were read using CellTiter-Glo to determine cell viability by quantifying ATP without virus infection. None of the 13 compounds showed toxicity, as the cell viability was above 80% for all compounds (FIG. 22D).

In the primary screening, halofantrine, amodiaquine, and chloroquine were identified as effective inhibitors of the SARS-COV2 JN.1 strain. We then conducted a dose-response curve assay to determine IC50 values at specified concentrations. IC50 calculations were performed using GraphPad Prism 10.1.0.

All three compounds exhibited IC50 values in the micromolar range, without cytotoxicity (FIG. 23A). Because amodiaquine is a Mannich base derivative related to chloroquine (Yamali, Gul et al. 2023), both showed similar IC50 values (11.95-12.14 μM) (FIG. 23B-Table 2). Halofantrine is a drug used to treat malaria, its structure contains a substituted phenanthrene, and is related to the antimalarial drugs quinine and lumefantrine (Sarhan, Ashour et al. 2021). It has been reported that halofantrine effectively binds to the main protease of SARS-COV-2 based on in silico analysis (Sachdeva, Wadhwa et al. 2020). In our cellular assays, halofantrine demonstrated activity against SARS-COV-2 JN.1 strain in the single-digit micromolar range, with an IC50 value of 4 μM (FIG. 23A). Additional research is needed to elucidate the mechanisms of action for halofantrine.

### Further Conclusions

The disclosed herein novel use of A549-Ace2-Dpp4-Tmprss2 human cell line exhibits high susceptibility to various coronaviruses, including SARS-COV-2, omicron subvariants, MERS-COV, OC43-CoV, and 229E-CoV. Analysis of viral replication demonstrates its applicability for antiviral testing and examining the broad-spectrum efficacy of compounds. A5498-ADT cells are easily cultured, possess a morphology conducive to imaging, and display cytopathic effects when infected with viruses such as SARS-COV-2, omicron subvariants, MERS-COV, and 229E-CoV. These cells can be infected as efficiently as VeroE6 cells and support viral replication to comparable high infectivity rates. Therefore, based on the results it is believed that innate immune response can be effectively activated by virus-infected A549-ADT cells making them more suitable for studying virus-host interactions.

A549-ADT cells have multiple benefits over Vero E6 cells. Firstly, as human lung cells, they provide a more physiologically relevant model for investigating single coronavirus infections or co-infections. Secondly, unlike Vero E6 cells, A549-ADT cells allow direct testing of compound antiviral activity without the need for P-gp efflux pump inhibitor co-treatment. Thirdly, robust imaging and CPE-based assays using A549-ADT cells yielded similar IC50 values for niamatrelvir as Vero E6 cells.

Lastly, A549-ADT cells facilitate the assessment and determination of antiviral activity across various human coronaviruses, including both alpha and beta coronaviruses. Although ensitrelvir showed no inhibitory effect on cells infected with 229E in our cell model, the engineered expression of Ace2, Dpp4, and Tmprss2 in A549-ADT cells may not accurately reflect the natural expression levels in human lung tissue, the of efficacy of ensitrelvir needs to be further studied using animal models. Moreover, the discovery of Halofantrine's antiviral activity against SARS-COV-2 opens up new possibilities for repurposing existing drugs to combat coronavirus infections.

In conclusion, it was found that the characterization of SARS-COV-2 and MERS-COV replication in A549-ADT cells, along with the described pan-coronavirus antiviral assays, provides a valuable cellular tool for studying lethal and seasonal coronaviruses and discovering broad-spectrum inhibitors of coronavirus replication.

Unless otherwise stated, the foregoing alternative examples are not mutually exclusive, but may be implemented in various combinations to achieve unique advantages. As these and other variations and combinations of the features discussed above can be utilized without departing from the subject matter defined by the claims, the foregoing description of the embodiments should be taken by way of illustration rather than by way of limitation of the subject matter defined by the claims. In addition, the provision of the examples described herein, as well as clauses phrased as “such as,” “including” and the like, should not be interpreted as limiting the subject matter of the claims to the specific examples; rather, the examples are intended to illustrate only one of many possible embodiments. Further, the same reference numbers in different drawings can identify the same or similar elements.

### SEQUENCE LISTING

SEQ. ID NO. 1:
ACE2
5′ATGTCAAGCTCTTCCTGGCTCCTTCTCAGCCTTGTTGCTGTAA
CTGCTGCTCAGTCCACCATTGAGGAACAGGCCAAGACATTTTTGG
ACAAGTTTAACCACGAAGCCGAAGACCTGTTCTATCAAAGTTCAC
TTGCTTCTTGGAATTATAACACCAATATTACTGAAGAGAATGTCC
AAAACATGAATAATGCTGGGGACAAATGGTCTGCCTTTTTAAAGG
AACAGTCCACACTTGCCCAAATGTATCCACTACAAGAAATTCAGA
ATCTCACAGTCAAGCTTCAGCTGCAGGCTCTTCAGCAAAATGGGT
CTTCAGTGCTCTCAGAAGACAAGAGCAAACGGTTGAACACAATTC
TAAATACAATGAGCACCATCTACAGTACTGGAAAAGTTTGTAACC
CAGATAATCCACAAGAATGCTTATTACTTGAACCAGGTTTGAATG
AAATAATGGCAAACAGTTTAGACTACAATGAGAGGCTCTGGGCTT
GGGAAAGCTGGAGATCTGAGGTCGGCAAGCAGCTGAGGCCATTAT
ATGAAGAGTATGTGGTCTTGAAAAATGAGATGGCAAGAGCAAATC
ATTATGAGGACTATGGGGATTATTGGAGAGGAGACTATGAAGTAA
ATGGGGTAGATGGCTATGACTACAGCCGCGGCCAGTTGATTGAAG
ATGTGGAACATACCTTTGAAGAGATTAAACCATTATATGAACATC
TTCATGCCTATGTGAGGGCAAAGTTGATGAATGCCTATCCTTCCT
ATATCAGTCCAATTGGATGCCTCCCTGCTCATTTGCTTGGTGATA
TGTGGGGTAGATTTTGGACAAATCTGTACTCTTTGACAGTTCCCT
TTGGACAGAAACCAAACATAGATGTTACTGATGCAATGGTGGACC
AGGCCTGGGATGCACAGAGAATATTCAAGGAGGCCGAGAAGTTCT
TTGTATCTGTTGGTCTTCCTAATATGACTCAAGGATTCTGGGAAA
ATTCCATGCTAACGGACCCAGGAAATGTTCAGAAAGCAGTCTGCC
ATCCCACAGCTTGGGACCTGGGGAAGGGCGACTTCAGGATCCTTA
TGTGCACAAAGGTGACAATGGACGACTTCCTGACAGCTCATCATG
AGATGGGGCATATCCAGTATGATATGGCATATGCTGCACAACCTT
TTCTGCTAAGAAATGGAGCTAATGAAGGATTCCATGAAGCTGTTG
GGGAAATCATGTCACTTTCTGCAGCCACACCTAAGCATTTAAAAT
CCATTGGTCTTCTGTCACCCGATTTTCAAGAAGACAATGAAACAG
AAATAAACTTCCTGCTCAAACAAGCACTCACGATTGTTGGGACTC
TGCCATTTACTTACATGTTAGAGAAGTGGAGGTGGATGGTCTTTA
AAGGGGAAATTCCCAAAGACCAGTGGATGAAAAAGTGGTGGGAGA
TGAAGCGAGAGATAGTTGGGGTGGTGGAACCTGTGCCCCATGATG
AAACATACTGTGACCCCGCATCTCTGTTCCATGTTTCTAATGATT
ACTCATTCATTCGATATTACACAAGGACCCTTTACCAATTCCAGT
TTCAAGAAGCACTTTGTCAAGCAGCTAAACATGAAGGCCCTCTGC
ACAAATGTGACATCTCAAACTCTACAGAAGCTGGACAGAAACTGT
TCAATATGCTGAGGCTTGGAAAATCAGAACCCTGGACCCTAGCAT
TGGAAAATGTTGTAGGAGCAAAGAACATGAATGTAAGGCCACTGC
TCAACTACTTTGAGCCCTTATTTACCTGGCTGAAAGACCAGAACA
AGAATTCTTTTGTGGGATGGAGTACCGACTGGAGTCCATATGCAG
ACCAAAGCATCAAAGTGAGGATAAGCCTAAAATCAGCTCTTGGAG
ATAAAGCATATGAATGGAACGACAATGAAATGTACCTGTTCCGAT
CATCTGTTGCATATGCTATGAGGCAGTACTTTTTAAAAGTAAAAA
ATCAGATGATTCTTTTTGGGGAGGAGGATGTGCGAGTGGCTAATT
TGAAACCAAGAATCTCCTTTAATTTCTTTGTCACTGCACCTAAAA
ATGTGTCTGATATCATTCCTAGAACTGAAGTTGAAAAGGCCATCA
GGATGTCCCGGAGCCGTATCAATGATGCTTTCCGTCTGAATGACA
ACAGCCTAGAGTTTCTGGGGATACAGCCAACACTTGGACCTCCTA
ACCAGCCCCCTGTTTCCATATGGCTGATTGTTTTTGGAGTTGTGA
TGGGAGTGATAGTGGTTGGCATTGTCATCCTGATCTTCACTGGGA
TCAGAGATCGGAAGAAGAAAAATAAAGCAAGAAGTGGAGAAAATC
CTTATGCCTCCATCGATATTAGCAAAGGAGAAAATAATCCAGGAT
TCCAAAACACTGATGATGTTCAGACCTCCTTTTAG3′
SEQ. ID NO. 2:
TMPRSS2
5′ATGGCTTTGAACTCAGGGTCACCACCAGCTATTGGACCTTACT
ATGAAAACCATGGATACCAACCGGAAAACCCCTATCCCGCACAGC
CCACTGTGGTCCCCACTGTCTACGAGGTGCATCCGGCTCAGTACT
ACCCGTCCCCCGTGCCCCAGTACGCCCCGAGGGTCCTGACGCAGG
CTTCCAACCCCGTCGTCTGCACGCAGCCCAAATCCCCATCCGGGA
CAGTGTGCACCTCAAAGACTAAGAAAGCACTGTGCATCACCTTGA
CCCTGGGGACCTTCCTCGTGGGAGCTGCGCTGGCCGCTGGCCTAC
TCTGGAAGTTCATGGGCAGCAAGTGCTCCAACTCTGGGATAGAGT
GCGACTCCTCAGGTACCTGCATCAACCCCTCTAACTGGTGTGATG
GCGTGTCACACTGCCCCGGCGGGGAGGACGAGAATCGGTGTGTTC
GCCTCTACGGACCAAACTTCATCCTTCAGGTGTACTCATCTCAGA
GGAAGTCCTGGCACCCTGTGTGCCAAGACGACTGGAACGAGAACT
ACGGGCGGGCGGCCTGCAGGGACATGGGCTATAAGAATAATTTTT
ACTCTAGCCAAGGAATAGTGGATGACAGCGGATCCACCAGCTTTA
TGAAACTGAACACAAGTGCCGGCAATGTCGATATCTATAAAAAAC
TGTACCACAGTGATGCCTGTTCTTCAAAAGCAGTGGTTTCTTTAC
GCTGTATAGCCTGCGGGGTCAACTTGAACTCAAGCCGCCAGAGCA
GGATCGTGGGCGGCGAGAGCGCGCTCCCGGGGGCCTGGCCCTGGC
AGGTCAGCCTGCACGTCCAGAACGTCCACGTGTGCGGAGGCTCCA
TCATCACCCCCGAGTGGATCGTGACAGCCGCCCACTGCGTGGAAA
AACCTCTTAACAATCCATGGCATTGGACGGCATTTGCGGGGATTT
TGAGACAATCTTTCATGTTCTATGGAGCCGGATACCAAGTAGAAA
AAGTGATTTCTCATCCAAATTATGACTCCAAGACCAAGAACAATG
ACATTGCGCTGATGAAGCTGCAGAAGCCTCTGACTTTCAACGACC
TAGTGAAACCAGTGTGTCTGCCCAACCCAGGCATGATGCTGCAGC
CAGAACAGCTCTGCTGGATTTCCGGGTGGGGGGCCACCGAGGAGA
AAGGGAAGACCTCAGAAGTGCTGAACGCTGCCAAGGTGCTTCTCA
TTGAGACACAGAGATGCAACAGCAGATATGTCTATGACAACCTGA
TCACACCAGCCATGATCTGTGCCGGCTTCCTGCAGGGGAACGTCG
ATTCTTGCCAGGGTGACAGTGGAGGGCCTCTGGTCACTTCGAAGA
ACAATATCTGGTGGCTGATAGGGGATACAAGCTGGGGTTCTGGCT
GTGCCAAAGCTTACAGACCAGGAGTGTACGGGAATGTGATGGTAT
TCACGGACTGGATTTATCGACAAATGAGGGCAGACGGCTAA3′
SEQ. ID. NO. 3:
DPP4
5′ATGAAGACACCGTGGAAGGTTCTTCTGGGACTGCTGGGTGCTG
CTGCGCTTGTCACCATCATCACCGTGCCCGTGGTTCTGCTGAACA
AAGGCACAGATGATGCTACAGCTGACAGTCGCAAAACTTACACTC
TAACTGATTACTTAAAAAATACTTATAGACTGAAGTTATACTCCT
TAAGATGGATTTCAGATCATGAATATCTCTACAAACAAGAAAATA
ATATCTTGGTATTCAATGCTGAATATGGAAACAGCTCAGTTTTCT
TGGAGAACAGTACATTTGATGAGTTTGGACATTCTATCAATGATT
ATTCAATATCTCCTGATGGGCAGTTTATTCTCTTAGAATACAACT
ACGTGAAGCAATGGAGGCATTCCTACACAGCTTCATATGACATTT
ATGATTTAAATAAAAGGCAGCTGATTACAGAAGAGAGGATTCCAA
ACAACACACAGTGGGTCACATGGTCACCAGTGGGTCATAAATTGG
CATATGTTTGGAACAATGACATTTATGTTAAAATTGAACCAAATT
TACCAAGTTACAGAATCACATGGACGGGGAAAGAAGATATAATAT
ATAATGGAATAACTGACTGGGTTTATGAAGAGGAAGTCTTCAGTG
CCTACTCTGCTCTGTGGTGGTCTCCAAACGGCACTTTTTTAGCAT
ATGCCCAATTTAACGACACAGAAGTCCCACTTATTGAATACTCCT
TCTACTCTGATGAGTCACTGCAGTACCCAAAGACTGTACGGGTTC
CATATCCAAAGGCAGGAGCTGTGAATCCAACTGTAAAGTTCTTTG
TTGTAAATACAGACTCTCTCAGCTCAGTCACCAATGCAACTTCCA
TACAAATCACTGCTCCTGCTTCTATGTTGATAGGGGATCACTACT
TGTGTGATGTGACATGGGCAACACAAGAAAGAATTTCTTTGCAGT
GGCTCAGGAGGATTCAGAACTATTCGGTCATGGATATTTGTGACT
ATGATGAATCCAGTGGAAGATGGAACTGCTTAGTGGCACGGCAAC
ACATTGAAATGAGTACTACTGGCTGGGTTGGAAGATTTAGGCCTT
CAGAACCTCATTTTACCCTTGATGGTAATAGCTTCTACAAGATCA
TCAGCAATGAAGAAGGTTACAGACACATTTGCTATTTCCAAATAG
ATAAAAAAGACTGCACATTTATTACAAAAGGCACCTGGGAAGTCA
TCGGGATAGAAGCTCTAACCAGTGATTATCTATACTACATTAGTA
ATGAATATAAAGGAATGCCAGGAGGAAGGAATCTTTATAAAATCC
AACTTAGTGACTATACAAAAGTGACATGCCTCAGTTGTGAGCTGA
ATCCGGAAAGGTGTCAGTACTATTCTGTGTCATTCAGTAAAGAGG
CGAAGTATTATCAGCTGAGATGTTCCGGTCCTGGTCTGCCCCTCT
ATACTCTACACAGCAGCGTGAATGATAAAGGGCTGAGAGTCCTGG
AAGACAATTCAGCTTTGGATAAAATGCTGCAGAATGTCCAGATGC
CCTCCAAAAAACTGGACTTCATTATTTTGAATGAAACAAAATTTT
GGTATCAGATGATCTTGCCTCCTCATTTTGATAAATCCAAGAAAT
ATCCTCTACTATTAGATGTGTATGCAGGCCCATGTAGTCAAAAAG
CAGACACTGTCTTCAGACTGAACTGGGCCACTTACCTTGCAAGCA
CAGAAAACATTATAGTAGCTAGCTTTGATGGCAGAGGAAGTGGTT
ACCAAGGAGATAAGATCATGCATGCAATCAACAGAAGACTGGGAA
CATTTGAAGTTGAAGATCAAATTGAAGCAGCCAGACAATTTTCAA
AAATGGGATTTGTGGACAACAAACGAATTGCAATTTGGGGCTGGT
CATATGGAGGGTACGTAACCTCAATGGTCCTGGGATCGGGAAGTG
GCGTGTTCAAGTGTGGAATAGCCGTGGCGCCTGTATCCCGGTGGG
AGTACTATGACTCAGTGTACACAGAACGTTACATGGGTCTCCCAA
CTCCAGAAGACAACCTTGACCATTACAGAAATTCAACAGTCATGA
GCAGAGCTGAAAATTTTAAACAAGTTGAGTACCTCCTTATTCATG
GAACAGCAGATGATAACGTTCACTTTCAGCAGTCAGCTCAGATCT
CCAAAGCCCTGGTCGATGTTGGAGTGGATTTCCAGGCAATGTGGT
ATACTGATGAAGACCATGGAATAGCTAGCAGCACAGCACACCAAC
ATATATATACCCACATGAGCCACTTCATAAAACAATGTTTCTCTT
TACCTTAG 3′

What is claimed is:

1. A method for assessment and validation of antivirals comprises:
receiving a sample collected from a test subject;
applying to the sample a genetically modified cell line of a A549-ACE2-DPP4-TMPRSS2 (ADT) cell line assay or a ACE2plusC3D4 model cell line assay that simultaneously express a three-receptor infection model receptors of SEQ ID NO: 1, SEQ ID NO: 2 and SEQ ID NO: 3 of angiotensin-converting enzyme 2 (ACE2), transmembrane serine protease 2 (TMPRSS2), and dipeptidyl peptidase 4 (DPP4), respectively; and
screening the sample with ADT or ACE2plusC3D4 having the three-receptor infection model receptors for a pan-human coronavirus.

2. The method of claim 1, wherein the screening further includes a prior adjustment of expression levels of the three-receptor infection model receptors for specific viral entry and infectivity for the cell line assay used for antiviral drug screening against pan-coronaviruses.

3. The method of claim 2, wherein the adjustment of expression levels include having a DPP4 expression level between 1.7 and 1.9 times greater than a control, and the screening includes at least one immunofluorescence test.

4. The method of claim 1, further includes studying virus-host interactions, and wherein application of the genetically modified cell line serves as a single cell line for multiple antiviral targets.

5. The method of claim 1, wherein the genetically modified cell line is susceptible to both a lethal coronavirus and a seasonal coronavirus, and wherein the pan-human coronavirus includes at least one of SARS-COV-2, MERS-COV, OC43-CoV, or 229E-CoV.

6. The method of claim 5, wherein the lethal coronavirus is OC43 and the seasonal coronavirus is 229E.

7. The method of claim 1, the A549-ADT assay with coronavirus yielded similar half-maximal inhibitory concentration (IC50) values for Nirmatrelvir as a VeroE6-based assay and wherein, the A549-ADT assay does not require a P-glycoprotein (P-gp) inhibitor to prevent drug efflux.

8. The method of claim 1, further includes conducting a visual imaging test and a cytopathic effect (CPE)-based assay.

9. The method of claim 8, further includes displaying visual CPE upon SARS-COV-2, MERS-COV or 229E-CoV infection to cells.

10. A method for assessment and validation of antivirals, comprises:
receiving a sample collected from a test subject;
applying to the sample a genetically modified cell line of a ACE2plusC3D4 model cell line assay or a A549-ACE2-DPP4-TMPRSS2 (ADT) cell line assay that simultaneously express a three-receptor infection model receptors of SEQ ID NO: 1, SEQ ID NO: 2 and SEQ ID NO: 3 of angiotensin-converting enzyme (ACE2), transmembrane serine protease 2 (TMPRSS2), and 2 dipeptidyl peptidase 4 (DPP4), respectively; and
screening the sample with the cell assay having the three-receptor infection model receptors for a pan-human coronavirus by at least one of an imaging test or a cytopathic effect (CPE)-based assay, and adjusting the expression levels of the three-receptor infection model receptors for specific viral entry and infectivity for the cell line assay used for antiviral drug screening against the pan-human coronavirus; and
wherein the imaging test and the CPE-based assay using the cell assay yields similar IC50 values for niamatrelvir as Vero E6 cells without the need for P-gp efflux pump inhibitor co-treatment.

11. The method of claim 10, the adjusting of expression levels include having a DPP4 expression level less than 2.0 times greater than a control, and the imaging test includes at least one immunofluorescence test.

12. The method of claim 10, wherein the A549-ADT cell assay provides a more physiologically relevant model for investigating single coronavirus infections or co-infections than Vero E6 cells, and the A549-ADT cell assay allows direct testing of compound antiviral activity.

13. The method of claim 10, wherein the A549-ADT cell assay facilitates an assessment and determination of antiviral activity across various human coronaviruses including both alpha and beta coronaviruses.

14. A kit assessment and validation of antivirals comprising:
a detection assay that includes a A549-ACE2-DPP4-TMPRSS2 (ADT) cell assay or a ACE2plusC3D4 model cell assay that simultaneously expresses a three-receptor infection model receptors of SEQ ID NO: 1, SEQ ID NO: 2 and SEQ ID NO: 3 of angiotensin-converting enzyme 2 (ACE2), transmembrane serine protease 2 (TMPRSS2), and dipeptidyl peptidase 4 (DPP4) for screening a sample for a pan-human coronavirus; and
wherein the cell assay has an adjustment of an expression level of the three-receptor infection model receptors for specific viral entry and infectivity for the assay used for antiviral drug screening against the pan-human coronavirus.

15. The kit of claim 14, wherein the adjustment of the expression level includes having a DPP4 expression level less than 2.0 times greater than a control, or the DPP4 expression level is between 1.7 and 1.9 times greater than the control.

16. The kit of claim 14, wherein the cell assay expresses ACE2 and TMPRSS2 and remains relatively stable to SARS-COV-2 infection susceptibility after multiple passages for performing high-throughput in vitro testing to evaluate efficacy of SARS-CoV-2 antivirals and facilitate research on drugs for COVID-19 treatment.

17. The kit of claim 14, wherein the cell assay is permissive to MERS-COV, SARS-COV-2 infection, and emerging omicron variants EG5.1, BQ1, XBB1.19 and XBB1.16.

18. The kit of claim 14, wherein virus-induced cytopathic effect (CPE) is inhibited by adding Niamatrelvir during virus infection, and the cell assay does not require a P-gp efflux pump inhibitor co-treatment unlike a Vero E6 cell detection assay.

19. The kit of claim 14, wherein the cell assay is used for studying coronavirus pathogenesis.

20. The kit of claim 14, wherein the cell assay is susceptible to both a lethal coronavirus and a seasonal coronavirus.


SEQUENCE LISTING (3 sequences)

SEQ ID NO: 1 - DNA, 2418 bp, synthetic construct
  source 1..2418: mol_type = other DNA
atgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggacgacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgtaaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttag

SEQ ID NO: 2 - DNA, 1479 bp, synthetic construct
  source 1..1479: mol_type = other DNA
atggctttgaactcagggtcaccaccagctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcaggtgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtatagcctgcggggtcaacttgaactcaagccgccagagcaggatcgtgggcggcgagagcgcgctcccgggggcctggccctggcaggtcagcctgcacgtccagaacgtccacgtgtgcggaggctccatcatcacccccgagtggatcgtgacagccgcccactgcgtggaaaaacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggctaa

SEQ ID NO: 3 - DNA, 2301 bp, synthetic construct
  source 1..2301: mol_type = other DNA
atgaagacaccgtggaaggttcttctgggactgctgggtgctgctgcgcttgtcaccatcatcaccgtgcccgtggttctgctgaacaaaggcacagatgatgctacagctgacagtcgcaaaacttacactctaactgattacttaaaaaatacttatagactgaagttatactccttaagatggatttcagatcatgaatatctctacaaacaagaaaataatatcttggtattcaatgctgaatatggaaacagctcagttttcttggagaacagtacatttgatgagtttggacattctatcaatgattattcaatatctcctgatgggcagtttattctcttagaatacaactacgtgaagcaatggaggcattcctacacagcttcatatgacatttatgatttaaataaaaggcagctgattacagaagagaggattccaaacaacacacagtgggtcacatggtcaccagtgggtcataaattggcatatgtttggaacaatgacatttatgttaaaattgaaccaaatttaccaagttacagaatcacatggacggggaaagaagatataatatataatggaataactgactgggtttatgaagaggaagtcttcagtgcctactctgctctgtggtggtctccaaacggcacttttttagcatatgcccaatttaacgacacagaagtcccacttattgaatactccttctactctgatgagtcactgcagtacccaaagactgtacgggttccatatccaaaggcaggagctgtgaatccaactgtaaagttctttgttgtaaatacagactctctcagctcagtcaccaatgcaacttccatacaaatcactgctcctgcttctatgttgataggggatcactacttgtgtgatgtgacatgggcaacacaagaaagaatttctttgcagtggctcaggaggattcagaactattcggtcatggatatttgtgactatgatgaatccagtggaagatggaactgcttagtggcacggcaacacattgaaatgagtactactggctgggttggaagatttaggccttcagaacctcattttacccttgatggtaatagcttctacaagatcatcagcaatgaagaaggttacagacacatttgctatttccaaatagataaaaaagactgcacatttattacaaaaggcacctgggaagtcatcgggatagaagctctaaccagtgattatctatactacattagtaatgaatataaaggaatgccaggaggaaggaatctttataaaatccaacttagtgactatacaaaagtgacatgcctcagttgtgagctgaatccggaaaggtgtcagtactattctgtgtcattcagtaaagaggcgaagtattatcagctgagatgttccggtcctggtctgcccctctatactctacacagcagcgtgaatgataaagggctgagagtcctggaagacaattcagctttggataaaatgctgcagaatgtccagatgccctccaaaaaactggacttcattattttgaatgaaacaaaattttggtatcagatgatcttgcctcctcattttgataaatccaagaaatatcctctactattagatgtgtatgcaggcccatgtagtcaaaaagcagacactgtcttcagactgaactgggccacttaccttgcaagcacagaaaacattatagtagctagctttgatggcagaggaagtggttaccaaggagataagatcatgcatgcaatcaacagaagactgggaacatttgaagttgaagatcaaattgaagcagccagacaattttcaaaaatgggatttgtggacaacaaacgaattgcaatttggggctggtcatatggagggtacgtaacctcaatggtcctgggatcgggaagtggcgtgttcaagtgtggaatagccgtggcgcctgtatcccggtgggagtactatgactcagtgtacacagaacgttacatgggtctcccaactccagaagacaaccttgaccattacagaaattcaacagtcatgagcagagctgaaaattttaaacaagttgagtacctccttattcatggaacagcagatgataacgttcactttcagcagtcagctcagatctccaaagccctggtcgatgttggagtggatttccaggcaatgtggtatactgatgaagaccatggaatagctagcagcacagcacaccaacatatatatacccacatgagccacttcataaaacaatgtttctctttaccttag